(19) United States
(12) Patent Application Publication
(10) Pub. No.: US 2026/0179779 A1
(43) Pub. Date: Jun. 25, 2026

(54) PERSONALIZED BENCHMARK AND HEALTH ANALYSIS

(71) Applicant: AiCare Corporation, San Jose, CA (US)

(72) Inventors: Fang Fang, Cupertino, CA (US); Xuzhe Zhi, Placentia, CA (US); Raymond Fangshuo Xu, Cupertino, CA (US); John Fee, Knoxville, TN (US)

(21) Appl. No.: 19/426,340

(22) Filed: Dec. 19, 2025

Related U.S. Application Data
(60) Provisional application No. 63/736,667, filed on Dec. 20, 2024.

(51) Int. Cl. G16H 50/30; G16H 10/40

(52) U.S. Cl. CPC G16H 50/30; G16H 10/40

(57) ABSTRACT

Provided herein are methods for determining a personalized risk assessment for a subject from one or more biometric data streams of the subject using a multimodal machine learning algorithm to establish personalized benchmark profile by: identifying for each of the normalized biometric data stream values for: minimum, maximum, delta, mean, median, and average minimum and average maximum boundaries; reducing noise from non-essential features by cross feature linear regression analysis to obtain a dimensionality reduction; and stratifying each of two or more normalized biometric data stream values into nodes with a high or maximum predictive value and minimum predictive value selected to calculate the personalized risk assessment; using nodes with high or maximum predictive value to identify biometric data values for the personalized risk assessment; and administering or conducting at least one of: post-disease recovery, post-procedure monitoring, diagnostic tests, or treatment to the subject based on the personalized risk assessment.

### CROSS-REFERENCE TO RELATED APPLICATIONS

This application claims priority to U.S. Provisional Application Ser. No. 63/736,667, filed Dec. 20, 2024, the entire contents of which are incorporated herein by reference.

### TECHNICAL FIELD OF THE INVENTION

The present invention relates in general to the field of wearable biometric device data to determine personalized health benchmark analysis, and more particularly, to machine learning algorithms and methods for improving the predictability and specificity of biometric data analysis.

### STATEMENT OF FEDERALLY FUNDED RESEARCH

None.

### INCORPORATION-BY-REFERENCE OF MATERIALS FILED ON COMPACT DISC

None.

### BACKGROUND OF THE INVENTION

Without limiting the scope of the invention, its background is described in connection with field of wearable biometric device data.

One such invention is disclosed in U.S. patent Ser. No. 12/106,313, issued to Advani, entitled “Generating Insights Based on Signals From Measuring Device”. This inventor is said to teach a measuring device that includes a sensor unit that generates weigh data, motion data, location data, and time consumed data of the product and transmits to a computing device. A communication device is then used to record the consumption or usage of the product by a user, along with feedback from the user. The computing device is also said to generate insights based on the sensed data generated by the measuring device and recording and feedback at the communication device.

Another such disclosure is taught in U.S. Patent Publication No. 20240347190, filed by Daniels, entitled, “Mask-Based Diagnostic Utilizing AI Algorithms For Improved Patient Outcomes.” This applicant is said to teach a mask-based diagnostic (MBD) system for remote patient monitoring that collects chemical biomarker data and non-chemical biometric data from patients in a non-invasive manner. The MBD is said to be used to monitor various medical conditions, including cardiovascular disease, lung cancer, diabetes, and respiratory diseases. The system is said to consists of a mask having an exhaled breath condensate (EBC) collector that tests for chemical biomarkers in EBC, as well as, non-chemical biometric data, such as temperature, heart rate, and blood oxygen levels can also be obtained using a wearable electronic device.

Despite these advances, a need remains for personalized biometric data analysis that can be used to make treatment decisions for a patient. Also needed are improvements to machine leaming/artificial intelligence models for analyzing and making sense to complex biometric data, and the use of the analysis to make treatment decisions.

### SUMMARY OF THE INVENTION

As embodied and broadly described herein, an aspect of the present disclosure relates to a method of determining a personalized risk assessment for a subject comprising: receiving and normalizing one or more biometric data streams of the subject; using a processor and a multimodal machine learning algorithm to establish personalized benchmark profile and determine personalized risk assessment: identifying for each of the normalized biometric data stream values for: minimum, maximum, delta, mean, median, and average minimum and average maximum boundaries; reducing noise from non-essential features by cross feature linear regression analysis to obtain a dimensionality reduction; and stratifying each of two or more normalized biometric data stream values into nodes with a high or maximum predictive value and minimum predictive value, wherein nodes with high or maximum predictive value are selected to calculate the personalized risk assessment; using the nodes with high or maximum predictive value to identify biometric data values for the personalized risk assessment; and administering conducting at least one of: post-disease recovery, post-procedure monitoring, diagnostic tests, or treatment to the subject based on the personalized risk assessment. In one aspect, the diagnostic tests are selected based on the personalized risk assessment selected from obtaining biological samples to test for bacterial, viral, fungal, or parasitic infection, metabolic panels, phenotyping panels, genotyping panels, microbiome panels, or autoimmune disease or condition testing. In another aspect, the treatment is selected from one or more cardiovascular drugs, antimicrobial drugs, wherein the disease, disorder or condition is an infectious disease or disorder, or an autoimmune disease. In another aspect, normalizing the one or more biometric data streams by categorizing the biometric data stream into three categories: maximum frequency biometric data, medium frequency biometric data, and minimum frequency biometric data and selecting data values for a fixed or variable time interval. In another aspect, the maximum frequency biometric data, medium frequency biometric data, and minimum frequency biometric data is selected from at least one of: daytime and nighttime, activity (1G, 2G, 3G acceleration), heart rate, heart rate variability, resting heart rate, blood oxygen saturation, or motion sensors that measure along at least one of an X, Y, or Z axis. In another aspect, the dimensionality reduction is selected from linear regression, z-score, p-value, gaussian, normal distribution, and statistical method. In another aspect, the method further comprises stratifying each of two or more normalized biometric data stream values into nodes with high or maximum predictive value and minimum predictive value. In another aspect, the method further comprises after using the nodes with high or maximum predictive value to identify biometric data values for the personalized risk assessment, then determining: if the one or more nodes meet or exceed a predetermined risk assessment value threshold, then using the nodes for the personalized risk assessment, or if the one or more nodes is below the predetermined risk assessment value threshold, then repeating the step of selecting one or more nodes using a different statistical model until the statistical significance meets or exceeds the personalized risk assessment threshold. In another aspect, the method further comprises, after using the nodes with high or maximum predictive value to identify biometric data values for the personalized risk assessment, then determining: if the one or more nodes meet or exceed a predetermined risk assessment value threshold, then using the nodes for the personalized risk assessment, or if the one or more nodes is below the predetermined risk assessment value threshold, then: repeating the step of receiving and normalizing one or more biometric data streams of the subject; repeating the step of identifying for each of the normalized biometric data stream values for: minimum, maximum, delta, mean, median, and average minimum and average maximum boundaries; repeating the step of reducing noise in each of the normalized biometric data stream values by: repeating the step of stratifying each of two or more normalized biometric data stream values into nodes with high or maximum or maximum predictive value and minimum predictive value, wherein nodes with high or maximum or maximum predictive value are selected to calculate the personalized risk assessment; or repeating the step of using the nodes with high or maximum predictive value to identify biometric data values for the personalized risk assessment, until: the statistical significance meets or exceeds the personalized risk assessment threshold. In another aspect, the one or more biometric data streams are obtained from a wearable device, one or more sensors transiently or permanently attached to or inserted into the subject, or sensors in a room, chair or bed. In another aspect, the method further comprises placing the subject into a cohort of patients with one or more medical conditions or diseases of the subject. In another aspect, the one or more data streams are obtained from at least one of: one or more wearable devices, one or more medical beds, one or more O2 sensors, one or more blood pressure sensors, one or more electrocardiogram (ECG), accelerometers, or gyroscopes. In another aspect, the one or more biometric sensor devices is selected from O2 sensor(s), accelerometer(s), gyroscope(s), electrocardiogram, accelerometer(s), gyroscope(s), heart rate monitor(s), or pulse monitor(s). In another aspect, the biometric data further identifies blood pressure, step count, active energy burned, basal energy burned, sleep status, temperature, respiratory rate, EKG, posture, or fall detection. In another aspect, the biometric data is categorized into three categories: high frequency, medium frequency, and low frequency; category I: high frequency with distinct patterns during day vs. night; category II: medium frequency data points with some daily data points; category III: low frequency with no or a few data points per day; and the personalized benchmark-based outliers, statistic-based features selected from at least one of: heart rate outliers: day upper outliers: the heart rate data points higher than an established day upper threshold of the subject; day lower outliers: the heart rate data points lower than an established day upper threshold of the subject; night upper outliers: the heart rate data points higher than an established day upper threshold of the subject; night lower outliers: the heart rate data points lower than an established day upper threshold of the subject; physical activities outlier-based features: at least one of: day 1 g, 2 g, or 3 g upper outliers; at least one of: day 1 g, 2 g, or 3 g lower outliers; at least one of: night 1 g, 2 g, or 3 g upper outliers; at least one of: night 1 g, 2 g, or 3 g lower outliers; and statistics: min, mean, median, max, sum. In another aspect, the one or more temporal segments in the Category I data streams are selected from 0.1, 1, 5, 10, 15, 20, 25, 30, 40, 50, 60, 70, 75, 80, 90, 100 milliseconds, 0.1, 1, 5, 10, 15, 20, 25, 30, 40, 50, 60, 70, 75, 80, 90, 100 seconds, 0.1, 1, 5, 10, 15, 20, 25, 30, 40, 50, 60, 70, 75, 80, 90, to 100 minutes. In another aspect, the one or more temporal segment in the Category II data streams are selected from 0.1, 1, 5, 10, 15, 20, 25, 30, 40, 50, 60, 70, 75, 80, 90, 100 hours, 0.1, 1, 5, 10, 15, 20, 25, 30, 40, 50, 60, 70, 75, 80, 90, 100 days, 0.1, 1, 5, 10, 15, 20, 25, 30, 40, 50, 52, 60, 70, 75, 80, 90, to 100 weeks. In another aspect, the one or more temporal segments in the Category III data streams are selected from 0.1, 1, 5, 10, 15, 20, 25, 30, 40, 50, 60, 70, 75, 80, 90, 100 days, 0.1, 1, 5, 10, 15, 20, 25, 30, 40, 50, 52, 60, 70, 75, 80, 90, 100 weeks, 0.1, 1, 5, 10, 15, 20, 25, 30, 40, 50, 60, 70, 75, 80, 90, to 100 months. In another aspect, the one or more temporal segments in the Category I, II, or III data streams can be fixed, variable, periodic, or aperiodic. In another aspect, the biometric data is selected from at least one of: nightmore3G_median; daymore1G_min; daymore2G_min; daymore3G_min; nightmore1G_min; nightmore1G_delta_min; nightmore1G_lower_outlier; nightmore1G_lower_outlier_percentage; nightmore2G_min; nightmore2G_delta_min; nightmore2G_lower_outlier; nightmore2G_lower_outlier_percentage; nightmore3G_min; nightmore3G_delta_min; nightmore3G_lower_outlier; nightmore3G_lower_outlier_percentage; daymore1G_delta_min; daymore1G_lower_outlier; daymore1G_lower_outlier_percentage; daymore2G_delta_min; daymore2G_lower_outlier; daymore2G_lower_outlier_percentage; daymore3G_delta_min; daymore3G_lower_outlier; daymore3G_lower_outlier_percentage; bloodOxygenSaturation_delta_max; bloodOxygenSaturation_upper_outlier; bloodOxygenSaturation_upper_outlier_percentage; nightmore2G_median; daymore3G_median; bloodOxygenSaturation_max; bloodOxygenSaturation_lower_outlier; bloodOxygenSaturation_outlier; dayheartRate_delta_min; bloodOxygenSaturation_delta_min; heartRateVariability_upper_outlier; dayheartRate_min; nightmore1G_median; heartRateVariability_lower_outlier_percentage; bloodOxygenSaturation_min; heartRateVariability_outlier; dayheartRate_max; nightmore1G_upper_outlier_percentage; nightmore1G_outlier_percentage; heartRateVariability_delta_max; nightmore1G_upper_outlier; nightmore1G_outlier; dayheartRate_delta_max; nightheartRate_min; restingHeartRate_upper_outlier_percentage; heartRateVariability_max; heartRateVariability_delta_min; nightheartRate_delta_min; restingHeartRate_delta_max; nightheartRate_mean; nightmore2G_delta_max; nightheartRate_upper_outlier_percentage; nightmore2G_max; dayheartRate_upper_outlier; nightheartRate_median; nightmore1G_delta_max; nightmore1G_max; restingHeartRate_max; heartRateVariability_lower_outlier; daymore3G_delta_max; restingHeartRate_median; daymore3G_max; dayheartRate_lower_outlier; restingHeartRate_upper_outlier; nightmore3G_mean; daymore2G_delta_max; nightheartRate_delta_max; nightheartRate_lower_outlier; daymore2G_max; restingHeartRate_mean; nightmore2G_mean; dayheartRate_outlier; nightmore3G_delta_max; dayheartRate_upper_outlier_percentage; nightmore3G_max; nightheartRate_max; daymore1G_delta_max; nightmore2G_upper_outlier_percentage; nightmore2G_outlier_percentage; nightmore1G_sum; nightmore3G_sum; nightmore2G_upper_outlier; nightmore2G_outlier; nightmore1G_mean; nightmore2G_sum; heartRateVariability_upper_outlier_percentage; nightheartRate_lower_outlier_percentage; nightheartRate_outlier; daymore2G_median; nightheartRate_upper_outlier; nightmore3G_upper_outlier_percentage; nightmore3G_outlier_percentage; daymore1G_upper_outlier; daymore1G_outlier; nightmore3G_upper_outlier; nightmore3G_outlier; restingHeartRate_lower_outlier; heartRateVariability_min; restingHeartRate_min; restingHeartRate_lower_outlier_percentage; bloodOxygenSaturation_median; heartRateVariability_outlier_percentage; daymore1G_max; daymore1G_sum; daymore1G_median; restingHeartRate_outlier; dayheartRate_median; daymore3G_upper_outlier; daymore3G_outlier; nightheartRate_outlier_percentage; daymore3G_sum; bloodOxygenSaturation_lower_outlier_percentage; bloodOxygenSaturation_outlier_percentage; dayheartRate_mean; restingHeartRate_delta_min; heartRateVariability_mean; heartRateVariability_median; daymore1G_upper_outlier_percentage; daymore1G_outlier_percentage; daymore3G_upper_outlier_percentage; daymore3G_outlier_percentage; daymore2G_sum; daymore3G_mean; daymore2G_upper_outlier; daymore2G_outlier; daymore1G_mean; dayheartRate_lower_outlier_percentage; daymore2G_mean; daymore2G_upper_outlier_percentage; daymore2G_outlier_percentage; bloodOxygenSaturation_mean; dayheartRate_outlier_percentage; or restingHeartRate_outlier_percentage.

As embodied and broadly described herein, an aspect of the present disclosure relates to a non-transitory computer-readable medium for determining a personalized risk assessment for a subject comprising instructions stored thereon, that when executed on a processor, perform the steps of: receiving an electronic communication containing one or more biometric data streams of the subject; using a processor and a multimodal machine learning algorithm to establish personalized benchmark profile and determine personalized risk assessment by: identifying for each of the normalized biometric data stream values for: minimum, maximum, delta, mean, median, and average minimum and average maximum boundaries; reducing noise from non-essential features by cross feature linear regression analysis to obtain a dimensionality reduction; and stratifying each of two or more normalized biometric data stream values into nodes with a high or maximum predictive value and minimum predictive value, wherein nodes with high or maximum predictive value are selected to calculate the personalized risk assessment; using the nodes with high or maximum predictive value to identify biometric data values for the personalized risk assessment; and administering conducting at least one of: post-disease recovery, post-procedure monitoring, diagnostic tests, or treatment to the subject based on the personalized risk assessment. In one aspect, the diagnostic tests are selected based on the personalized risk assessment selected from obtaining biological samples to test for bacterial, viral, fungal, or parasitic infection, metabolic panels, phenotyping panels, genotyping panels, microbiome panels, or autoimmune disease or condition testing. In another aspect, the treatment is selected from one or more cardiovascular drugs, antimicrobial drugs, wherein the disease, disorder or condition is an infectious disease or disorder, or an autoimmune disease. In another aspect, normalizing the one or more biometric data streams by categorizing the biometric data stream into three categories: maximum frequency biometric data, medium frequency biometric data, and minimum frequency biometric data and selecting data values for a fixed or variable time interval. In another aspect, the maximum frequency biometric data, medium frequency biometric data, and minimum frequency biometric data is selected from at least one of: daytime and nighttime, activity (1G, 2G, 3G acceleration), heart rate, heart rate variability, resting heart rate, blood oxygen saturation, or motion sensors that measure along at least one of an X, Y, or Z axis. In another aspect, the dimensionality reduction is selected from linear regression, z-score, p-value, gaussian, normal distribution, and statistical method. In another aspect, the method further comprises stratifying each of two or more normalized biometric data stream values into nodes with high or maximum predictive value and minimum predictive value. In another aspect, the method further comprises after using the nodes with high or maximum predictive value to identify biometric data values for the personalized risk assessment, then determining: if the one or more nodes meet or exceed a predetermined risk assessment value threshold, then using the nodes for the personalized risk assessment, or if the one or more nodes is below the predetermined risk assessment value threshold, then repeating the step of selecting one or more nodes using a different statistical model until the statistical significance meets or exceeds the personalized risk assessment threshold. In another aspect, the method further comprises, after using the nodes with high or maximum predictive value to identify biometric data values for the personalized risk assessment, then determining: if the one or more nodes meet or exceed a predetermined risk assessment value threshold, then using the nodes for the personalized risk assessment, or if the one or more nodes is below the predetermined risk assessment value threshold, then: repeating the step of receiving and normalizing one or more biometric data streams of the subject; repeating the step of identifying for each of the normalized biometric data stream values for: minimum, maximum, delta, mean, median, and average minimum and average maximum boundaries; repeating the step of reducing noise in each of the normalized biometric data stream values by: repeating the step of stratifying each of two or more normalized biometric data stream values into nodes with high or maximum or maximum predictive value and minimum predictive value, wherein nodes with high or maximum or maximum predictive value are selected to calculate the personalized risk assessment; or repeating the step of using the nodes with high or maximum predictive value to identify biometric data values for the personalized risk assessment, until: the statistical significance meets or exceeds the personalized risk assessment threshold. In another aspect, the one or more biometric data streams are obtained from a wearable device, one or more sensors transiently or permanently attached to or inserted into the subject, or sensors in a room, chair or bed. In another aspect, the method further comprises placing the subject into a cohort of patients with one or more medical conditions or diseases of the subject. In another aspect, the one or more data streams are obtained from at least one of: one or more wearable devices, one or more medical beds, one or more O2 sensors, one or more blood pressure sensors, one or more electrocardiogram (ECG), accelerometers, or gyroscopes. In another aspect, the one or more biometric sensor devices is selected from O2 sensor(s), accelerometer(s), gyroscope(s), electrocardiogram, accelerometer(s), gyroscope(s), heart rate monitor(s), or pulse monitor(s). In another aspect, the biometric data further identifies blood pressure, step count, active energy burned, basal energy burned, sleep status, temperature, respiratory rate, EKG, posture, or fall detection. In another aspect, the biometric data is categorized into three categories: high frequency, medium frequency, and low frequency; category I: high frequency with distinct patterns during day vs. night; category II: medium frequency data points with some daily data points; category III: low frequency with no or a few data points per day; and the personalized benchmark-based outliers, statistic-based features selected from at least one of: heart rate outliers: day upper outliers: the heart rate data points higher than an established day upper threshold of the subject; day lower outliers: the heart rate data points lower than an established day upper threshold of the subject; night upper outliers: the heart rate data points higher than an established day upper threshold of the subject; night lower outliers: the heart rate data points lower than an established day upper threshold of the subject; physical activities outlier-based features: at least one of: day 1 g, 2 g, or 3 g upper outliers; at least one of: day 1 g, 2 g, or 3 g lower outliers; at least one of: night 1 g, 2 g, or 3 g upper outliers; at least one of: night 1 g, 2 g, or 3 g lower outliers; and statistics: min, mean, median, max, sum. In another aspect, the one or more temporal segments in the Category I data streams are selected from 0.1, 1, 5, 10, 15, 20, 25, 30, 40, 50, 60, 70, 75, 80, 90, 100 milliseconds, 0.1, 1, 5, 10, 15, 20, 25, 30, 40, 50, 60, 70, 75, 80, 90, 100 seconds, 0.1, 1, 5, 10, 15, 20, 25, 30, 40, 50, 60, 70, 75, 80, 90, to 100 minutes. In another aspect, the one or more temporal segment in the Category II data streams are selected from 0.1, 1, 5, 10, 15, 20, 25, 30, 40, 50, 60, 70, 75, 80, 90, 100 hours, 0.1, 1, 5, 10, 15, 20, 25, 30, 40, 50, 60, 70, 75, 80, 90, 100 days, 0.1, 1, 5, 10, 15, 20, 25, 30, 40, 50, 52, 60, 70, 75, 80, 90, to 100 weeks. In another aspect, the one or more temporal segments in the Category III data streams are selected from 0.1, 1, 5, 10, 15, 20, 25, 30, 40, 50, 60, 70, 75, 80, 90, 100 days, 0.1, 1, 5, 10, 15, 20, 25, 30, 40, 50, 52, 60, 70, 75, 80, 90, 100 weeks, 0.1, 1, 5, 10, 15, 20, 25, 30, 40, 50, 60, 70, 75, 80, 90, to 100 months. In another aspect, the one or more temporal segments in the Category I, II, or III data streams can be fixed, variable, periodic, or aperiodic. In another aspect, the biometric data is selected from at least one of: nightmore3G_median; daymore1G_min; daymore2G_min; daymore3G_min; nightmore1G_min; nightmore1G_delta_min; nightmore1G_lower_outlier; nightmore1G_lower_outlier_percentage; nightmore2G_min; nightmore2G_delta_min; nightmore2G_lower_outlier; nightmore2G_lower_outlier_percentage; nightmore3G_min; nightmore3G_delta_min; nightmore3G_lower_outlier; nightmore3G_lower_outlier_percentage; daymore1G_delta_min; daymore1G_lower_outlier; daymore1G_lower_outlier_percentage; daymore2G_delta_min; daymore2G_lower_outlier; daymore2G_lower_outlier_percentage; daymore3G_delta_min; daymore3G_lower_outlier; daymore3G_lower_outlier_percentage; bloodOxygenSaturation_delta_max; bloodOxygenSaturation_upper_outlier; bloodOxygenSaturation_upper_outlier_percentage; nightmore2G_median; daymore3G_median; bloodOxygenSaturation_max; bloodOxygenSaturation_lower_outlier; bloodOxygenSaturation_outlier; dayheartRate_delta_min; bloodOxygenSaturation_delta_min; heartRateVariability_upper_outlier; dayheartRate_min; nightmore1G_median; heartRateVariability_lower_outlier_percentage; bloodOxygenSaturation_min; heartRateVariability_outlier; dayheartRate_max; nightmore1G_upper_outlier_percentage; nightmore1G_outlier_percentage; heartRateVariability_delta_max; nightmore1G_upper_outlier; nightmore1G_outlier; dayheartRate_delta_max; nightheartRate_min; restingHeartRate_upper_outlier_percentage; heartRateVariability_max; heartRateVariability_delta_min; nightheartRate_delta_min; restingHeartRate_delta_max; nightheartRate_mean; nightmore2G_delta_max; nightheartRate_upper_outlier_percentage; nightmore2G_max; dayheartRate_upper_outlier; nightheartRate_median; nightmore1G_delta_max; nightmore1G_max; restingHeartRate_max; heartRateVariability_lower_outlier; daymore3G_delta_max; restingHeartRate_median; daymore3G_max; dayheartRate_lower_outlier; restingHeartRate_upper_outlier; nightmore3G_mean; daymore2G_delta_max; nightheartRate_delta_max; nightheartRate_lower_outlier; daymore2G_max; restingHeartRate_mean; nightmore2G_mean; dayheartRate_outlier; nightmore3G_delta_max; dayheartRate_upper_outlier_percentage; nightmore3G_max; nightheartRate_max; daymore1G_delta_max; nightmore2G_upper_outlier_percentage; nightmore2G_outlier_percentage; nightmore1G_sum; nightmore3G_sum; nightmore2G_upper_outlier; nightmore2G_outlier; nightmore1G_mean; nightmore2G_sum; heartRateVariability_upper_outlier_percentage; nightheartRate_lower_outlier_percentage; nightheartRate_outlier; daymore2G_median; nightheartRate_upper_outlier; nightmore3G_upper_outlier_percentage; nightmore3G_outlier_percentage; daymore1G_upper_outlier; daymore1G_outlier; nightmore3G_upper_outlier; nightmore3G_outlier; restingHeartRate_lower_outlier; heartRateVariability_min; restingHeartRate_min; restingHeartRate_lower_outlier_percentage; bloodOxygenSaturation_median; heartRateVariability_outlier_percentage; daymore1G_max; daymore1G_sum; daymore1G_median; restingHeartRate_outlier; dayheartRate_median; daymore3G_upper_outlier; daymore3G_outlier; nightheartRate_outlier_percentage; daymore3G_sum; bloodOxygenSaturation_lower_outlier_percentage; bloodOxygenSaturation_outlier_percentage; dayheartRate_mean; restingHeartRate_delta_min; heartRateVariability_mean; heartRateVariability_median; daymore1G_upper_outlier_percentage; daymore1G_outlier_percentage; daymore3G_upper_outlier_percentage; daymore3G_outlier_percentage; daymore2G_sum; daymore3G_mean; daymore2G_upper_outlier; daymore2G_outlier; daymore1G_mean; dayheartRate_lower_outlier_percentage; daymore2G_mean; daymore2G_upper_outlier_percentage; daymore2G_outlier_percentage; bloodOxygenSaturation_mean; dayheartRate_outlier_percentage; or restingHeartRate_outlier_percentage. In another aspect, a circadian boundary detection used hourly heart rate and acceleration via k means. In another aspect, the method further comprises determining an outlier counting value per circadian segment, wherein biometric outliers are counted separately for each morning segment, daytime segment and night segment relative to one or more personalized thresholds. In another aspect, the method further comprises using a dynamic benchmark window to generates a dynamic physiological benchmark by combining an initialization window comprising an early segment of user data with a rolling, continuously updated benchmark window, to provide one or more flexible and progressively personalized comparison metrics for risk detection. In another aspect, the method further comprises using one or more nodes comprising one or more related features to evaluate and stratify the data using one or more statistical tests selected from t tests or z scores to determine a predictive strength. In another aspect, the method further comprises calculating one or more personalized feature contribution scores that computes a relative weight of one or more biometric data or datastreams during short-term or long-term conditions to rank features by overall impact on predictions across a part of or all of a dataset.

As embodied and broadly described herein, an aspect of the present disclosure relates to a computer-implemented method for determining a personalized risk assessment for a subject, the method comprising: receiving an electronic communication containing one or more biometric data streams of the subject; normalizing the dataset; using a processor and a multimodal machine learning algorithm to establish personalized benchmark profile and determine personalized risk assessment: identifying for each of the normalized biometric data stream values for: minimum, maximum, delta, mean, median, and average minimum and average maximum boundaries; reducing noise from non-essential features by cross feature linear regression analysis to obtain a dimensionality reduction; and stratifying each of two or more normalized biometric data stream values into nodes with a high or maximum predictive value and minimum predictive value, wherein nodes with high or maximum predictive value are selected to calculate the personalized risk assessment; using the nodes with high or maximum predictive value to identify biometric data values for the personalized risk assessment; and administering conducting at least one of: post-disease recovery, post-procedure monitoring, diagnostic tests, or treatment to the subject based on the personalized risk assessment. In one aspect, the diagnostic tests are selected based on the personalized risk assessment selected from obtaining biological samples to test for bacterial, viral, fungal, or parasitic infection, metabolic panels, phenotyping panels, genotyping panels, microbiome panels, or autoimmune disease or condition testing. In another aspect, the treatment is selected from one or more cardiovascular drugs, antimicrobial drugs, wherein the disease, disorder or condition is an infectious disease or disorder, or an autoimmune disease. In another aspect, normalizing the one or more biometric data streams by categorizing the biometric data stream into three categories: maximum frequency biometric data, medium frequency biometric data, and minimum frequency biometric data and selecting data values for a fixed or variable time interval. In another aspect, the maximum frequency biometric data, medium frequency biometric data, and minimum frequency biometric data is selected from at least one of: daytime and nighttime, activity (1G, 2G, 3G acceleration), heart rate, heart rate variability, resting heart rate, blood oxygen saturation, or motion sensors that measure along at least one of an X, Y, or Z axis. In another aspect, the dimensionality reduction is selected from linear regression, z-score, p-value, gaussian, normal distribution, and statistical method. In another aspect, the method further comprises stratifying each of two or more normalized biometric data stream values into nodes with high or maximum predictive value and minimum predictive value. In another aspect, the method further comprises after using the nodes with high or maximum predictive value to identify biometric data values for the personalized risk assessment, then determining: if the one or more nodes meet or exceed a predetermined risk assessment value threshold, then using the nodes for the personalized risk assessment, or if the one or more nodes is below the predetermined risk assessment value threshold, then repeating the step of selecting one or more nodes using a different statistical model until the statistical significance meets or exceeds the personalized risk assessment threshold. In another aspect, the method further comprises, after using the nodes with high or maximum predictive value to identify biometric data values for the personalized risk assessment, then determining: if the one or more nodes meet or exceed a predetermined risk assessment value threshold, then using the nodes for the personalized risk assessment, or if the one or more nodes is below the predetermined risk assessment value threshold, then: repeating the step of receiving and normalizing one or more biometric data streams of the subject; repeating the step of identifying for each of the normalized biometric data stream values for: minimum, maximum, delta, mean, median, and average minimum and average maximum boundaries; repeating the step of reducing noise in each of the normalized biometric data stream values by: repeating the step of stratifying each of two or more normalized biometric data stream values into nodes with high or maximum or maximum predictive value and minimum predictive value, wherein nodes with high or maximum or maximum predictive value are selected to calculate the personalized risk assessment; or repeating the step of using the nodes with high or maximum predictive value to identify biometric data values for the personalized risk assessment, until: the statistical significance meets or exceeds the personalized risk assessment threshold. In another aspect, the one or more biometric data streams are obtained from a wearable device, one or more sensors transiently or permanently attached to or inserted into the subject, or sensors in a room, chair or bed. In another aspect, the method further comprises placing the subject into a cohort of patients with one or more medical conditions or diseases of the subject. In another aspect, the one or more data streams are obtained from at least one of: one or more wearable devices, one or more medical beds, one or more O2 sensors, one or more blood pressure sensors, one or more electrocardiogram (ECG), accelerometers, or gyroscopes. In another aspect, the one or more biometric sensor devices is selected from O2 sensor(s), accelerometer(s), gyroscope(s), electrocardiogram, accelerometer(s), gyroscope(s), heart rate monitor(s), or pulse monitor(s). In another aspect, the biometric data further identifies blood pressure, step count, active energy burned, basal energy burned, sleep status, temperature, respiratory rate, EKG, posture, or fall detection. In another aspect, the biometric data is categorized into three categories: high frequency, medium frequency, and low frequency; category I: high frequency with distinct patterns during day vs. night; category II: medium frequency data points with some daily data points; category III: low frequency with no or a few data points per day; and the personalized benchmark-based outliers, statistic-based features selected from at least one of: heart rate outliers: day upper outliers: the heart rate data points higher than an established day upper threshold of the subject; day lower outliers: the heart rate data points lower than an established day upper threshold of the subject; night upper outliers: the heart rate data points higher than an established day upper threshold of the subject; night lower outliers: the heart rate data points lower than an established day upper threshold of the subject; physical activities outlier-based features: at least one of: day 1 g, 2 g, or 3 g upper outliers; at least one of: day 1 g, 2 g, or 3 g lower outliers; at least one of: night 1 g, 2 g, or 3 g upper outliers; at least one of: night 1 g, 2 g, or 3 g lower outliers; and statistics: min, mean, median, max, sum. In another aspect, the one or more temporal segments in the Category I data streams are selected from 0.1, 1, 5, 10, 15, 20, 25, 30, 40, 50, 60, 70, 75, 80, 90, 100 milliseconds, 0.1, 1, 5, 10, 15, 20, 25, 30, 40, 50, 60, 70, 75, 80, 90, 100 seconds, 0.1, 1, 5, 10, 15, 20, 25, 30, 40, 50, 60, 70, 75, 80, 90, to 100 minutes. In another aspect, the one or more temporal segment in the Category II data streams are selected from 0.1, 1, 5, 10, 15, 20, 25, 30, 40, 50, 60, 70, 75, 80, 90, 100 hours, 0.1, 1, 5, 10, 15, 20, 25, 30, 40, 50, 60, 70, 75, 80, 90, 100 days, 0.1, 1, 5, 10, 15, 20, 25, 30, 40, 50, 52, 60, 70, 75, 80, 90, to 100 weeks. In another aspect, the one or more temporal segments in the Category III data streams are selected from 0.1, 1, 5, 10, 15, 20, 25, 30, 40, 50, 60, 70, 75, 80, 90, 100 days, 0.1, 1, 5, 10, 15, 20, 25, 30, 40, 50, 52, 60, 70, 75, 80, 90, 100 weeks, 0.1, 1, 5, 10, 15, 20, 25, 30, 40, 50, 60, 70, 75, 80, 90, to 100 months. In another aspect, the one or more temporal segments in the Category I, II, or III data streams can be fixed, variable, periodic, or aperiodic. In another aspect, the biometric data is selected from at least one of: nightmore3G_median; daymore1G_min; daymore2G_min; daymore3G_min; nightmore1G_min; nightmore1G_delta_min; nightmore1G_lower_outlier; nightmore1G_lower_outlier_percentage; nightmore2G_min; nightmore2G_delta_min; nightmore2G_lower_outlier; nightmore2G_lower_outlier_percentage; nightmore3G_min; nightmore3G_delta_min; nightmore3G_lower_outlier; nightmore3G_lower_outlier_percentage; daymore1G_delta_min; daymore1G_lower_outlier; daymore1G_lower_outlier_percentage; daymore2G_delta_min; daymore2G_lower_outlier; daymore2G_lower_outlier_percentage; daymore3G_delta_min; daymore3G_lower_outlier; daymore3G_lower_outlier_percentage; bloodOxygenSaturation_delta_max; bloodOxygenSaturation_upper_outlier; bloodOxygenSaturation_upper_outlier_percentage; nightmore2G_median; daymore3G_median; bloodOxygenSaturation_max; bloodOxygenSaturation_lower_outlier; bloodOxygenSaturation_outlier; dayheartRate_delta_min; bloodOxygenSaturation_delta_min; heartRateVariability_upper_outlier; dayheartRate_min; nightmore1G_median; heartRateVariability_lower_outlier_percentage; bloodOxygenSaturation_min; heartRateVariability_outlier; dayheartRate_max; nightmore1G_upper_outlier_percentage; nightmore1G_outlier_percentage; heartRateVariability_delta_max; nightmore1G_upper_outlier; nightmore1G_outlier; dayheartRate_delta_max; nightheartRate_min; restingHeartRate_upper_outlier_percentage; heartRateVariability_max; heartRateVariability_delta_min; nightheartRate_delta_min; restingHeartRate_delta_max; nightheartRate_mean; nightmore2G_delta_max; nightheartRate_upper_outlier_percentage; nightmore2G_max; dayheartRate_upper_outlier; nightheartRate_median; nightmore1G_delta_max; nightmore1G_max; restingHeartRate_max; heartRateVariability_lower_outlier; daymore3G_delta_max; restingHeartRate_median; daymore3G_max; dayheartRate_lower_outlier; restingHeartRate_upper_outlier; nightmore3G_mean; daymore2G_delta_max; nightheartRate_delta_max; nightheartRate_lower_outlier; daymore2G_max; restingHeartRate_mean; nightmore2G_mean; dayheartRate_outlier; nightmore3G_delta_max; dayheartRate_upper_outlier_percentage; nightmore3G_max; nightheartRate_max; daymore1G_delta_max; nightmore2G_upper_outlier_percentage; nightmore2G_outlier_percentage; nightmore1G_sum; nightmore3G_sum; nightmore2G_upper_outlier; nightmore2G_outlier; nightmore1G_mean; nightmore2G_sum; heartRateVariability_upper_outlier_percentage; nightheartRate_lower_outlier_percentage; nightheartRate_outlier; daymore2G_median; nightheartRate_upper_outlier; nightmore3G_upper_outlier_percentage; nightmore3G_outlier_percentage; daymore1G_upper_outlier; daymore1G_outlier; nightmore3G_upper_outlier; nightmore3G_outlier; restingHeartRate_lower_outlier; heartRateVariability_min; restingHeartRate_min; restingHeartRate_lower_outlier_percentage; bloodOxygenSaturation_median; heartRateVariability_outlier_percentage; daymore1G_max; daymore1G_sum; daymore1G_median; restingHeartRate_outlier; dayheartRate_median; daymore3G_upper_outlier; daymore3G_outlier; nightheartRate_outlier_percentage; daymore3G_sum; bloodOxygenSaturation_lower_outlier_percentage; bloodOxygenSaturation_outlier_percentage; dayheartRate_mean; restingHeartRate_delta_min; heartRateVariability_mean; heartRateVariability_median; daymore1G_upper_outlier_percentage; daymore1G_outlier_percentage; daymore3G_upper_outlier_percentage; daymore3G_outlier_percentage; daymore2G_sum; daymore3G_mean; daymore2G_upper_outlier; daymore2G_outlier; daymore1G_mean; dayheartRate_lower_outlier_percentage; daymore2G_mean; daymore2G_upper_outlier_percentage; daymore2G_outlier_percentage; bloodOxygenSaturation_mean; dayheartRate_outlier_percentage; or restingHeartRate_outlier_percentage.

### BRIEF DESCRIPTION OF THE DRAWINGS

For a more complete understanding of the features and advantages of the present invention, reference is now made to the detailed description of the invention along with the accompanying figures and in which:

FIG. 1 is a flowchart that shows an overview of the personalized benchmark and health analysis for circadian rhythm, biometrics and physical activities of the present invention. Module 1 (on the right) is for circadian rhythm identification. Module 2 (on the left) is for personalized benchmark establishment and precise risk prediction.

FIG. 2 is a graph that shows a sample subject's physical activities for a full day. X-axis shows the 0-24 hour of a day. For the Y-axis, scale on the right shows the physical activities, and scale on the left shows the sleep status as labels. When the subject moved at night, the sleep status transitioned from asleep (1.0) to inbed (0.5). This transition correlates with the physical activities' peaks.

FIG. 3 is a graph that shows another sample subject's physical activities for a full day.

FIG. 4 is a graph that shows a plot of physical activities hourly mean (y-axis) along 24 hours for a given day (x-axis).

FIG. 5 is a graph that shows a plot of physical activities hourly min (x-axis) and hourly max (y-axis).

FIG. 6 is a graph that shows a sample subject's physical activities and heart rate for a full day. X-axis shows the 0-24 hour of a day. For the Y-axis, scale on the right shows the heart rate, and scale on the left shows the sleep status as labels. This figure visualizes physical activities and heart rate trends between the day and night periods.

FIG. 7 is a graph that shows a plot of heart rate hourly mean (y-axis) along 24 hours for a given day (x-axis).

FIG. 8 is a graph that shows a plot of heart rate hourly mean (x-axis) and physical activities hourly mean (y-axis).

FIG. 9 is a graph that shows a Joined 2-Mean on multiple features. X-axis represents heart rate; y-axis represents physical activities. Color represents predicted clusters.

FIG. 10 is a graph that shows a 5-Mean on multiple features. X-axis represents physical activities; y-axis represents heart rate. Color represents predicted clusters.

FIG. 11 is a graph that shows a K-Nearest Neighbors experimentation. X-axis represents heart rate; y-axis represents physical activities. Color represents prediction results on sleep vs. non-sleep time.

FIG. 12 is a graph that shows a K-Mean analysis. X-axis represents heart rate, and y-axis represents physical activities. Color represents prediction results on sleep (yellow) vs. non-sleep time (purple).

FIG. 13 is a graph that shows a sample sleep analysis when subject did not wear Wearable device at night

FIG. 15 is a graph that shows a sleep pattern week-over-week changes for patient 1.

FIG. 16 is a graph that shows a sleep pattern week-over-week changes for patient 2.

FIG. 17 is a graph that shows illustrates the asleep and awake distribution across all 70+ patients. It is based on weekly aggregation. It tolerates missing data entries for a few days.

FIG. 18 is a graph that shows a heart rate distribution for patient d0eabf86fa from February 18 to February 28, first 10 days into the trial. The diagram illustrates a highly skewed distribution.

FIG. 19 is a graph that shows a sample heart rate personalized benchmark. X-axis represents time of the day. Y-axis represents heart rate measurements. The personalized upper benchmark is 119 and lower benchmark is 72.

FIG. 20 are graphs that shows daily blood oxygen saturation graphs with personal benchmark and outliers colored in red. The personalized upper benchmark is 100 and lower benchmark is 88, whereas the clinical threshold is 100 and 93.

FIG. 21 is a graph that shows a long-term blood oxygen saturation graph. The purple lines represent the personalized benchmarks, and the green lines represent the clinical thresholds.

FIG. 22A to 22C shows a long-term blood oxygen saturation outlier report, a daily outlier report, and an hourly outlier report. FIG. 22A the x-axis represents each day; y-axis represents outlier count for the given day. For this specific example, it shows the data points for patient ID 63504f0771 for the clinical trial duration. FIG. 22B shows daily outlier count of the heart rate incidents (low outliers and high outliers) based on personalized benchmark thresholds for daytime segment and nighttime segment. FIG. 22C shows hourly outlier count of the physical activities (>=1 gravity force) based on personalized benchmark thresholds for daytime segment and nighttime segment.

FIG. 23 is a graph that shows a personalized day/night 1G distribution over 3 patients (d3cea1dbaa, f92c105431, 0507df7ada).

FIG. 24 is a graph that shows a heart rate variability distribution for 3 patients (d3cea1dbaa, f92c105431, 0507df7ada).

FIG. 25 shows the correlation value of different subjects of different features. Each column represents a different subject ID; each row represents an input feature.

FIG. 26 shows the results from one sample t test over the correlation values for a subjects' cohort (subset of the subjects under study). Each column represents t test statistic result in different directions (feature value increasing or decreasing over time). Each row represents an input feature (label on the right).

FIG. 27 is a pie chart that summarized the sample subjects cohorts. In the above diagram, the overall study population is categorized into three cohorts: 13.3%, 17.3% and 69.3% respectively. The subjects in different cohorts demonstrate different characteristics in the study.

FIG. 28 is a diagram that illustrates that cohort 1 is not clearly separated from the total subject population. It shows the distribution of the cost value for the p-value based model.

FIG. 29 show the distribution of cost value for the z-score based model. Compared with the p-value based model, this model improves on the separation of cohort 1 from the total subject population. Train cohort 1: cohort 1 subjects' population in the training dataset. Test cohort 1: cohort 1 subjects' population in the test dataset. Test non-cohort 1: those subjects that do not belong to cohort 1 in the test dataset.

FIG. 30 shows a linear regression analysis between distanceWalkingRunning and stepCount. System reduces feature distanceWalkingRunning and keeps stepCount because they're highly correlated.

FIG. 31 shows a linear regression analysis between heartRate and stepCount. System keeps both of these features because their correlation is not significantly high.

FIG. 32 shows the sample feature selection. The features will be considered in the model if more than a given percentage of subjects in the cohort (70% in this example) share the trend in the same direction. In this illustration, selected features are dayheartRate_median and dayheartRate_outlier_percentage.

FIG. 33 shows a correlation scores for a given feature and a given subject. Each column represents one subject. Each row represents one feature.

FIG. 34 shows the correlation between heart-related features. Each square represents the value of correlation (with deep red indicating values close to 1 and deep blue indicating values close to −1). Features belonging to the same biomarkers usually show stronger correlation (groups of red squares near the diagonal). Some relationships are also observed between features of different biomarkers. For example, the mean, max, and median of heart rate variability are strongly negatively correlated with night heart rate-related features and resting heart rate-related features (blue squares).

FIG. 35 shows the correlation heat map between blood Oxygen related features.

FIG. 36 shows the correlation heatmap among physical activity (more1G, more2G, more3G) related features. Features for night physical activities are strongly positively correlated with each other. Night more1G features are weakly positively correlated with day physical activities. However, night more2G and night more3G features are negatively correlated with day physical activity features.

FIG. 37 is a flowchart that illustrates the explainability of the ML model which is a requirement or strongly preferred feature in the healthcare domain. The higher the feature in the decision process, in general, it carries more weight. The colors indicate whether the majority of samples in the subgroup at this stage of the tree belong to Group 1 (blue) or Group 2 (orange). The higher intensity of the color indicates a greater proportion of the subgroup belonging to one group compared to the other. A more intense color signifies a higher purity of the node, meaning most samples in that node belong to a single group, while a lighter color indicates a more mixed distribution of samples from both groups.

FIG. 38 is a graph that shows the use of the risk score prediction was able to identify an episode 4 days prior to the need for a medical intervention, the x-axis represents the day, y-axis represents the risk score. The higher the score, the higher the risk.

FIG. 39 shows three graphs that include AiCare's risk prediction based on a personalized benchmark prior to diagnosis with COVID.

FIG. 40 shows the results of the AiCare AI model was applied to a total of 90+ long-COVID patients to predict individuals' clinical improvement. The dataset was based on wearable data. Model performance: sensitivity: 95.83% (˜96%), specificity: 86.67% (˜87%), accuracy: 93.65%.

FIG. 41 summarizes 6 major long-COVID symptoms from a multi-patient analysis using the present invention.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

This patent outlines innovative approaches to leveraging at-home wearable device data to activate personalized health benchmark analysis, highlight the changes, predict the risks, and reduce the alarm fatigue. The documented approaches have been proven effective in multiple FDA approved clinical trials.

For example, disclosed herein is a novel, three-tier frequency normalization. Briefly, the three tier frequency normalization is a method that divides biometric data streams into three frequency tiers (high or maximum, medium, low or minimum) for structured normalization and processing. A high frequency or maximum tier will include data that is generally captured many times per second, minute, or hour, such as the continuous monitoring of an electrocardiogram (ECG), SpO₂, respiration, or pulse. The medium tier of frequency are those data streams that are acquired in a time frame from minutes to hours, such as, blood pressure, ECGs, or arrythmias. Finally, the low or minimum tier of frequency of data streams are those that are hourly, a few times daily, daily, or weekly, such as biochemical, pathogenic organism (bacteria, viral, or other pathogens), MRI, CAT scans, etc.

Also disclosed herein is a circadian boundary detection method using hourly heart rate and acceleration via k means. The hourly heart rate and acceleration via k means technique computes hourly averages of heart rate and acceleration, then applies k means clustering to automatically identify day night physiological boundaries.

Next, disclosed herein is a method for outlier counting per circadian segment. Briefly, the outlier counting per circadian segment includes as a feature an engineering approach where biometric outliers are counted separately for each segment (morning, daytime, night) relative to personalized thresholds.

Also disclosed is the use of a Dynamic Benchmark Window (DBW). DBW is a method that generates a dynamic physiological benchmark by combining an initialization window comprising the early segment of user data with a rolling, continuously updated benchmark window, enabling flexible and progressively personalized comparison metrics used for risk detection. More particularly, the DBW can by automatically or user programmable during the initialization window, so the benchmark will be established using the mentoring parameters from the device or electronic medical records (EMR) record (for example) or other data sources which are not part of the monitoring devices used. As used herein, the term “early segment” refers to an initial or subsequent data segment (e.g., 2^{nd}, 3^{rd}, 4^{th}, etc.) that is before later segments, which the skilled artisan will understand may vary depending on the frequency with which the data segment is gathered, that is, is thousands of data points are collected per minute or hour (high or maximum frequency), then the data segment may be in the first 0 to 5 percent of thousands of segments or data points. If the data segments are gathered every minute, few minutes, or hourly, then the data segment may be after a few, tens, or dozens, again within that initial 0 to 10 percent. Finally, if the data segments are hourly, daily, or weekly, again the earliest data segments may be after one or two days, but generally, following the initial 0 to 15 percent of data segments.

For example, an advanced monitoring system can provide continuous, cuff-less tracking of key indicators such as, e.g., blood pressure (BP), SpO2, respiration, pulse, electrocardiogram (ECG), posture, and life-threatening arrhythmias. Monitoring can take place over multi-hour or multi-days, or can be user programmed in any different time period that depends on the frequency of the biometric data streams.

Also disclosed herein is the use of node stratification using statistical tests. In this example, the nodes are composed of related features or datastreams that are evaluated and stratified using statistical tests such as t tests or z scores to determine predictive strength.

Using one or more of the above, a Personalized Feature Contribution Score (PFCS) is determined. The PFCS is a personalized feature-contribution scoring system that computes the relative weight of each biometric feature during short-term or long-term conditions, enabling individualized interpretability independent from global feature importance methods such as Shapley Additive exPlanations (SHAP). One example is the use of dynamic node reweighting or reselection. Dynamic node reweighting or reselection is a method that monitors node performance and automatically reweights or replaces nodes when their predictive value declines. While the Dynamic node reweighting or reselection can be automated or based on pre-determined parameters, it is also possible provide for user input-based Dynamic node reweighting or reselection. In user-based Dynamic node reweighting or reselection, the user is able to make decisions and set one or more thresholds about the extent, limits, boundaries, of influence of certain markers on the Dynamic node reweighting or reselection process and/or output.

Finally, disclosed herein is an iterative model significance threshold loop. The iterative model significance threshold loop is a system that repeatedly evaluates model significance thresholds and automatically adjusts feature sets or models until acceptable predictive quality is achieved. As with the Dynamic node reweighting or reselection, the iterative model significance threshold loop can be automated or based on pre-determined parameters, it is also possible provide for user input-based Dynamic node reweighting or reselection. In user-based iterative model significance threshold loop, the user is able to make decisions and set one or more thresholds about the extent, limits, boundaries, of influence of certain markers on the iterative model significance threshold loop process and/or output.

As used herein, a “higher frequency” of data points refers to data stream that is captured both day and night at least once every second, minute, hour, or day with distinct patterns during day vs. night. Non-limiting examples of higher frequency data stream for use with the present invention include: heart rate, or physical activity. A higher frequency data stream includes data that is obtained every 1 to 60 milliseconds, 1 to 60 seconds, 1 to 60 minutes, or 1 to 8 hours.

As used herein, a “medium frequency” of data points refer to a data stream that is captured daily. Non-limiting examples of medium frequency data stream for use with the present invention include a data stream with enough data points to determine from heart rate variability, or resting heart rate. A medium frequency data stream includes data that is obtained every 1 to 8 times a day, three times a day, twice a day, or daily.

As used herein, a “low frequency” of data points refers to a data stream with none, or a few data points per day. Non-limiting examples of low frequency data stream for use with the present invention include data streams that are obtained at least weekly selected from manually triggered ECG measure. A low frequency data stream includes data that is obtained every daily, every other day, three to six times a week, weekly, or monthly.

This disclosure introduces three systems.

System 1 is the circadian rhythm identification using the novel circadian ML prediction algorithms. It introduces algorithms and techniques to segment temporal biometric data into distinct day and night segments based on the identified personalized circadian boundaries.

System 2 is the personalized benchmark establishment, catering to various biometric patterns and outliers' analysis during day and night periods. The system activates precise and personalized health condition analysis and risk prediction.

System 3: AI-Based Personalized Health Risk Prediction Using Wearable Data: AI Model Building.

Precision Case Enablement via Personalization: this patent emphasizes personalized health analysis to individual biometric patterns, activating accurate health monitoring and risk prediction solutions.

Extensibility on Use Cases: the system is adaptable to a wide range of wearable data depending on the availability of the biometrics data for a given use case, expanding the system's usability. The data includes, but not limited to, heart rate, heart rate variability, resting heart rate, oxygen saturation, blood pressure, physical activities, step count, active energy burned, basal energy burned, and sleep status, etc.

Enhanced Solution Adoption: By addressing outliers such as occasional missing wearable data, the system strengthens the solution adoption.

Architecture Diagram. Following architecture diagram shows an overview of the systems. The workflow starts from module 1 (on the right) for circadian rhythm identification. Then the day/night boundary is streamed to module 2 (on the left) for temporal biometrics data segmentation, personalized benchmark establishment, and precise health condition analysis and risk prediction.

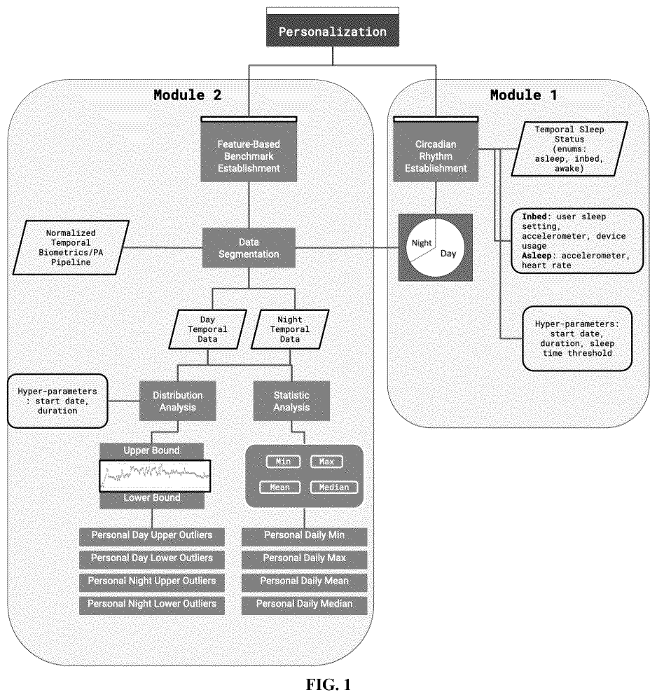

FIG. 1. Personalized benchmark and health analysis for circadian rhythm, biometrics and physical activities. Module 1 (on the right) is for circadian rhythm identification. Module 2 (on the left) is for personalized benchmark establishment and precise risk prediction.

### System 1: Circadian Rhythm ML Prediction

To achieve the risk prediction accuracy, the inventors segmented the wearable temporal biometric features into day and night segments because they exhibit different thresholds and patterns. Besides the daily based data segmentation, the long term trend of the day and night boundary for a given subject, nap in day time, activities at night time are important signals to risk prediction AI models. More particularly, personalized day and night thresholds are those that can use separate physiological baselines for day and night periods to improve model interpretability and stability. Thile much research has been done on circadian baselines exist, personalized day and night thresholds have not been applied risk model thresholds, alone, or in combination with other measurements as disclosed herein. Moreover, the day/night segmentation grouping features are included in predictive nodes. These day/night segmentation grouping features can be included in predictive nodes, e.g., clustering or grouping correlated biometric features into higher level predictive units called nodes. Selecting high predictive nodes via statistical significance involves choosing nodes with strong predictive relationships by applying statistical tests such as correlation or p value analysis.

There are sleep tracking products on the market. However, most of the algorithms are proprietary and the underlying raw data based on which the algorithms were developed is not publicly accessible. Instead of asking platform users to purchase and wear another commercially available device to track sleep, the inventors developed a circadian algorithm using the same wearable that is used to predict risk, however, the present invention can use any data platform/wearable device as a source of data. By being agnostic as to data platform/wearable device the approach of the present invention reduces both the cost and complexity for the subjects.

The method of actigraphy to estimate sleep only relies on movement data and is not able to detect certain wake moments at night. To overcome this issue, the inventors developed multi-dimensional biometric feature algorithms to detect circadian rhythm.

Dataset. The present invention used acceleration (in g-force unit: 9.8 m/s2), heartrate (bpm), and sleep status from wearable devices to identify circadian rhythm. The AI model is extensible to incorporate other biometric features, such as temperature, blood pressure.

Specific to the experimentation result mentioned in this patent, the data was collected using each data entry is associated with a unique code per subject. The personal identifiable information is not present in the dataset. Typical frequency of the data features is shown below. It depends on the sensor devices and subjects' movement status, etc. The average data frequency of the acceleration data is around 1 data point/minute. When the subject is in exercise, the frequency increases up to a few hundred milliseconds. The average data frequency of the heart rate data in general is 3-7 minutes. It's also observed at higher frequencies, such as in a few seconds.

TABLE 1
Sample heart rate wearable data in raw data format

| createUtc | createLocalTime | startUtc | heartRate |
|---|---|---|---|
| 05-15 | 05-15T17:05:54.080 | 05-15 | 86 |
| 05-15 | 05-15T17:08:09.082 | 05-15 | 83 |
| 05-15 | 05-15T17:13:34.080 | 05-15 | 64 |
| 05-15 | 05-15T17:19:35.081 | 05-15 | 70 |
| 05-15 | 05-15T17:20:25.967 | 05-15 | 67 |
| 05-15 | 05-15T17:21:38.830 | 05-15 | 76 |
| 05-15 | 05-15T17:28:12.832 | 05-15 | 90 |
| . . . | . . . | . . . | . . . |
| 05-15 | 05-15T18:20:06.708 | 05-15 | 172 |
| 05-15 | 05-15T18:20:09.708 | 05-15 | 173 |
| 05-15 | 05-15T18:20:15.708 | 05-15 | 175 |
| 05-15 | 05-15T18:20:21.708 | 05-15 | 177 |
| 05-15 | 05-15T18:20:27.708 | 05-15 | 179 |
| 05-15 | 05-15T18:20:32.708 | 05-15 | 180 |
| 05-15 | 05-15T18:20:36.708 | 05-15 | 180 |
| 05-15 | 05-15T18:20:42.708 | 05-1 | 181 |

Wearable device detects and sends sleep status in categorical values: asleep, inBed, awake.

InBed: wearable device considers user sleep setting, accelerometer, device usage.

Asleep: wearable device considers accelerometer, heart rate.

TABLE 2
Sample sleep analysis wearable data in
raw data format from Wearable device.

| createLocalTime | sleepAnalysis |
|---|---|
| 04-19 00:44:58 | asleep |
| 04-19 00:57:58 | asleep |
| 04-19 01:09:28 | asleep |
| 04-19 01:18:58 | asleep |
| 04-19 01:34:28 | asleep |
| . . . | . . . |
| 04-21 07:49:44 | awake |
| 04-21 07:51:14 | inBed |
| 04-21 07:53:14 | awake |
| 04-21 07:54:44 | inBed |
| 04-21 07:56:04 | inBed |

Data Availability Compliance. The subjects are required to wear the devices 75% of the time per day (with 6 hours for charging and some buffer time), and 4 days of the week (minimum 3 workdays and 1 weekend). The daily wear should cover both day and night time.

### Data Preprocessing and Normalization

First, remove the following disqualified subjects. Those who violate the data availability compliance. Those who experiment less than the benchmark period (e.g. 10 days). Those who travel to another time zone during personalized benchmark establishment time, or more than 10% of the total experimentation time. Then, the acceleration data and heart rate data are normalized to a consistent interval (e.g. 1, 300, 60, 3,600 seconds depending on the use cases). The 3-dimensional acceleration data (x, y, z) is normalized to 1 dimension.

### Circadian Machine Learning (ML) Prediction Algorithms.

The inventors developed 3 approaches as shown below. The following illustration is based on three input features: acceleration, heart rate and sleep status. The AI model framework is extensible to incorporate other biometric features, such as temperature, blood pressure.

For approach 1 and 2 where the system does not receive sleep status from the subject wearable device, the system then uses available biometrics data to infer circadian rhythm. To validate the model performance, the inventors used sleep status as labels to cross check the circadian rhythm model accuracy.

For approach 3 the system gets biometrics data as well as sleep status data from the subject wearable device, the inventors used both biometrics data and sleep status data as input features for model development to enhance the accuracy.

### Approach 1—Acceleration-Based Analysis.

Data Plot. The following figures show physical activities for a sample subject for a full day. The physical activity measures are used as the input feature to training the ML model. The inventors used sleep status from a wearable device as a benchmark label to evaluate the model accuracy. Sleep status labels are categorical values. The inventors used the categorical values “asleep” and “inbed” as training labels.

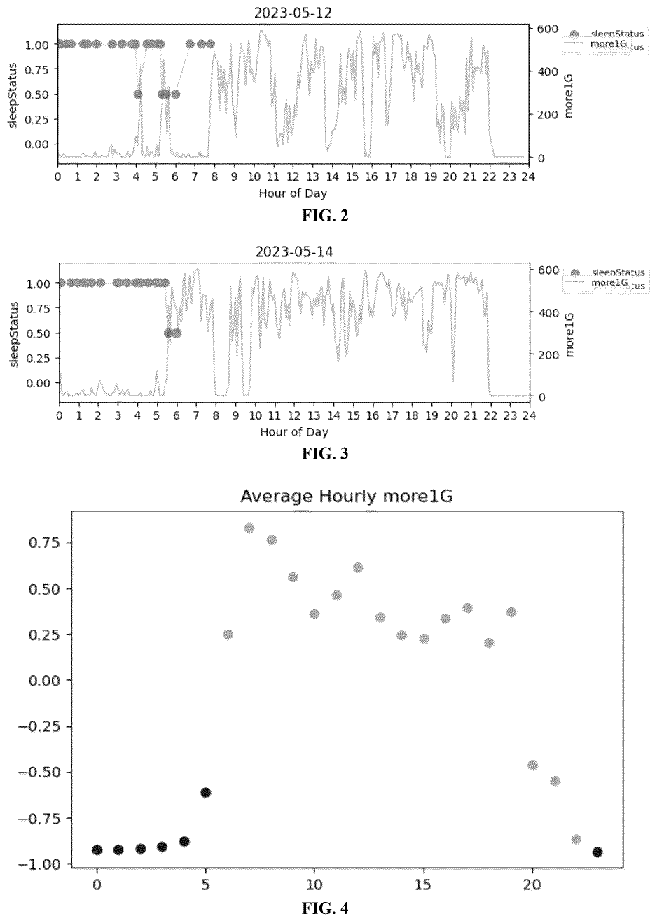

FIG. 2. Sample subject's physical activities for a full day. X-axis shows the 0-24 hour of a day. For the Y-axis, scale on the right shows the physical activities, and scale on the left shows the sleep status as labels. When the subject moved at night, the sleep status transitioned from asleep (1.0) to inbed (0.5). This transition correlates with the physical activities' peaks.

FIG. 3. Another sample subject's physical activities for a full day.

Intuition of Physical Activities Correlation with Sleep Labels. The inventors calculated the physical activities' hourly mean, hourly max and hourly min. In order to get intuition and visually observe the correlation between physical activities statistic values with the sleep labels, the inventors plotted the data in one graph.

First, the inventors plotted the hourly mean, and color-coded the values with labels (sleep time in blue and non-sleep time in orange).

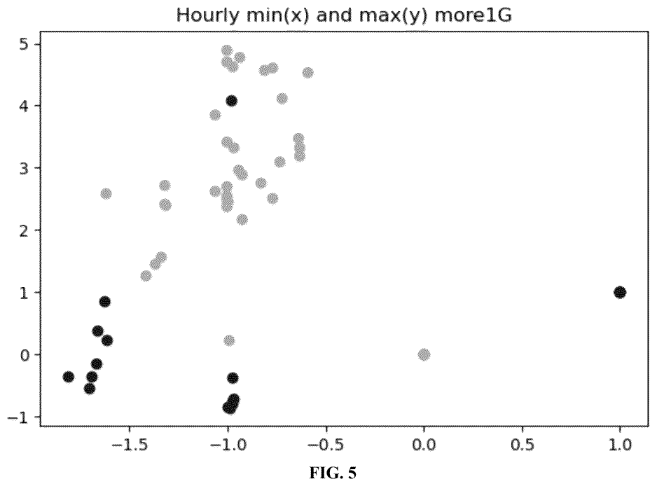

FIG. 4: Plot of physical activities hourly mean (y-axis) along 24 hours for a given day (x-axis).

The sleep labels are color coded with sleep time in blue and non-sleep time in orange. This graph visualizes the correlation between physical activities hourly mean and sleep labels.

Then, the inventors plotted the hourly min and max, and color-coded the values with labels (sleep time in blue and non-sleep time in orange).

FIG. 5: Plot of physical activities hourly min (x-axis) and hourly max (y-axis).

The sleep labels are color coded with sleep time in blue and non-sleep time in orange. This graph visualizes the correlation between hourly min/max and sleep labels.

Based on computation, the hourly mean of physical activities show stronger correlation with sleep status.

### Approach 2—Acceleration and Heart Rate-Based Analysis.

Data Plot. The following figures show physical activities and heart rate for a sample subject for a full day. The heart rate measures are used as the input feature to training the ML model. The inventors used sleep status from Wearable device as a benchmark label to evaluate the model accuracy. Sleep status labels are categorical values. The inventors used the categorical values “asleep” and “inbed” as training labels.

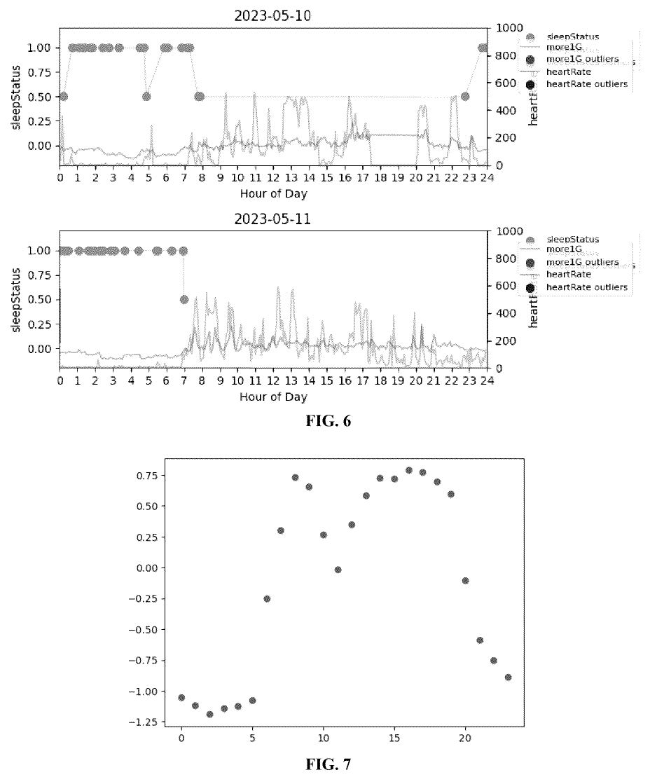

FIG. 6: Sample subject's physical activities and heart rate for a full day. X-axis shows the 0-24 hour of a day. For the Y-axis, scale on the right shows the heart rate, and scale on the left shows the sleep status as labels. This figure visualizes physical activities and heart rate trends between the day and night periods.

Intuition of Physical Activities and Heart Rate Correlation with Sleep Labels

First, the inventors calculated the heart rate' hourly mean and plotted the data in the graph.

FIG. 7: Plot of heart rate hourly mean (y-axis) along 24 hours for a given day (x-axis).

Then, the inventors plotted the hourly mean of both physical activities and heart rate, and color-coded the values with labels (sleep time in blue and non-sleep time in orange).

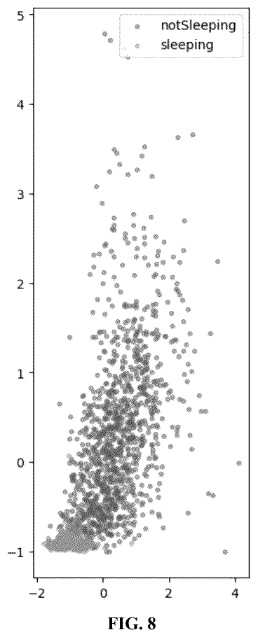

FIG. 8: Plot of heart rate hourly mean (x-axis) and physical activities hourly mean (y-axis).

The sleep labels are color coded with sleep time in blue and non-sleep time in orange. This graph visualizes the correlation between the hourly mean of physical activities, heart rate and sleep labels.

### Approach 3—Acceleration, Heart Rate, and Sleep Status-Based Analysis

If subjects provide sleep status as part of the wearable data, the system adjusts to approach 3, which uses sleep status as part of the input feature to enhance the accuracy.

### ML Prediction Results Analysis.

The inventors experimented and tuned different algorithms: 2-Mean, 5-Means clustering on one feature, on multiple features, and K-Nearest Neighbors. One example of a selection is K-Means on hourly mean values. In one example, the circadian boundary detection uses an hourly heart rate and acceleration via k means, which is a technique that computes hourly averages of heart rate and acceleration, then applies k means clustering to automatically identify day night physiological boundaries.

Following graphs illustrate 2-Mean, 5-Means and K-Nearest Neighbors.

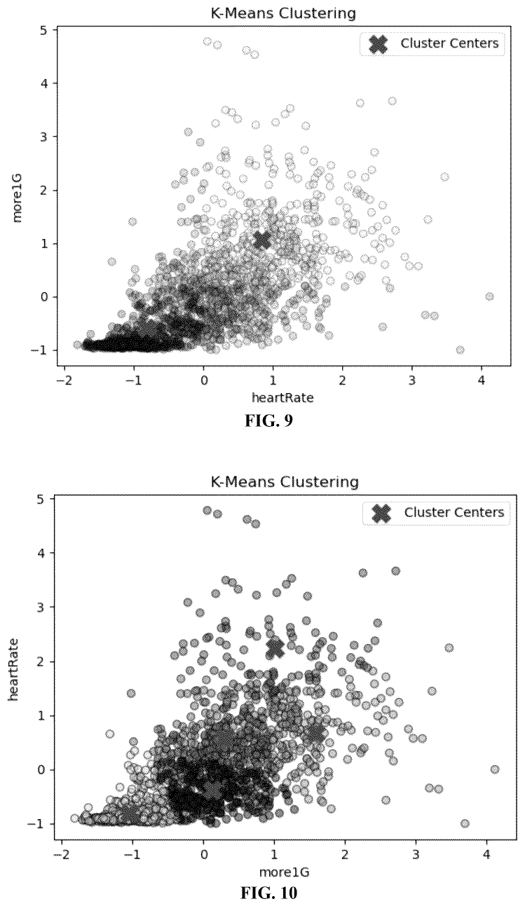

FIG. 9: Joined 2-Mean on multiple features. X-axis represents heart rate; y-axis represents physical activities. Color represents predicted clusters.

FIG. 10: 5-Mean on multiple features. X-axis represents physical activities; y-axis represents heart rate. Color represents predicted clusters.

FIG. 11: K-Nearest Neighbors experimentation. X-axis represents heart rate; y-axis represents physical activities. Color represents prediction results on sleep vs. non-sleep time.

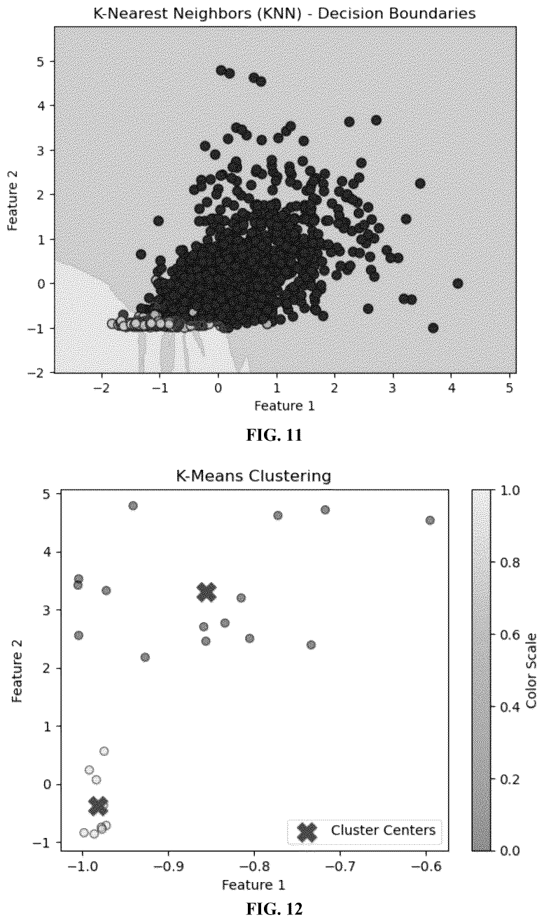

FIG. 12: K-Mean analysis. X-axis represents heart rate, and y-axis represents physical activities. Color represents prediction results on sleep (yellow) vs. non-sleep time (purple).

The best selection algorithm for this data was shown to be the 2-Mean on hourly mean of Heart Rate and Physical Activities.

TABLE 3
Circadian ML Model Precision Result

| | 2-Mean on Heart | Joined 2-Mean on | 5-Mean on Heart |
|---|---|---|---|
| | Rate and | Heart Rate and | Rate and |
| | Physical | Physical | Physical |
| | Activities | Activities | Activities |
| Accuracy | 0.9167 | 0.7822 | 0.4768 |
| Precision | 0.7778 | 0.5339 | 0.9728 |
| Sensitivity/ | 1.0000 | 0.9921 | 0.3118 |
| Recall | | | |
| Specificity | 0.8824 | 0.7125 | 0.9738 |

### Handling the Outliers.

Outlier Scenarios. Occasional missing data points, e.g., missing data at night.

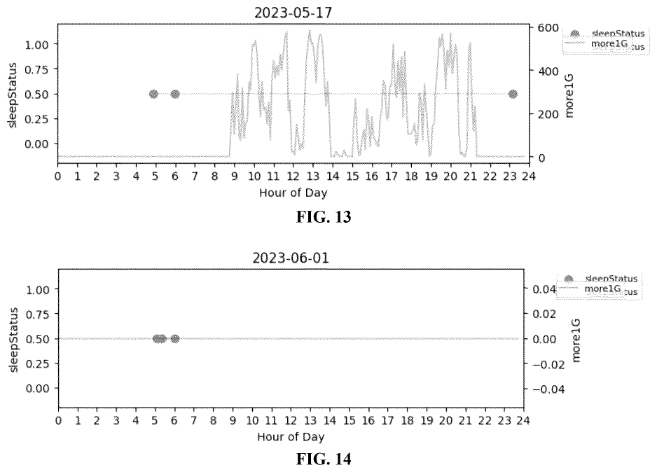

FIG. 13. Sample sleep analysis when subject did not wear Wearable device at night.

Wearable device runs out of battery or in charging.

FIG. 14. Sample sleep analysis when the Wearable device is not in use for the whole day. Therefore, the inventors only observed inBed status without asleep and awake. This diagram further proves that the inBed status is dependent on phone setting.

Subjects take nap(s) during the daytime, we've observed this especially inpatients and senior subjects.

In a trial, the inventors observed daytime sleep status for fatigue patients.

### Solutions to Handle the Outliers.

The inventors defined the following hyper-parameters to address the above mentioned outliers in order to enhance the analysis accuracy and solution adoption:

Define the time window to generate the personal benchmark. This period of days is configured as hyper-parameters. By default, the system sets it to 10 days, but it can be adjusted as needed.

Aggregate across a period of days to account for the missing data points for a few days in between.

This is to address outlier's scenario 1 (occasional missing data) and scenario 2 (run out of battery).

Benchmark start date, e.g. start of the clinical trial.

Benchmark duration, e.g. 10 days

Variable depending on the disease types, ranging from 1 day to 30 days

E.g. heart failure recovering patients/long-COVID patients: 10 days

E.g. knee replacement patients: 15 days

E.g. chronicle disease: 30 days

Set sleep time threshold to address outlier scenario 3 (nap) mentioned above.

Use rolling mean algorithm to differentiate sleep vs. short time nap.

If the input features do not have sleep analysis, the system can use physical activities and heart rate information to infer the circadian rhythm.

### Day/Night Boundary Results.

Based on the above algorithms, the inventors derived the personalized day and night boundary as circadian rhythm. Here are some examples of the established benchmark results.

TABLE 4
Computed circadian rhythm for sample subjects. The
values presented in the table are time of the day.

| Patient ID | Wake | Sleep |
|---|---|---|
| c967a93563 | 6.78 | 24 |
| ded5e8bc6d | 6.69 | 22.11 |
| e645b19ebf | 7.42 | 24 |
| 56b9f1b58a | 10.83 | 20.4 |
| c50abbb4f2 | 5.29 | 23 |
| 3a87eb0165 | 7.64 | 22.73 |

Following two diagrams demonstrate the trend of the personalized circadian rhythm.

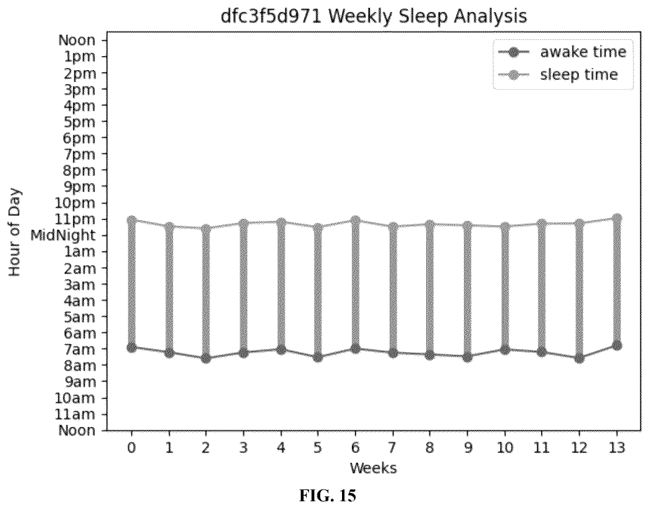

FIG. 15. Sleep pattern week-over-week changes for patient 1.

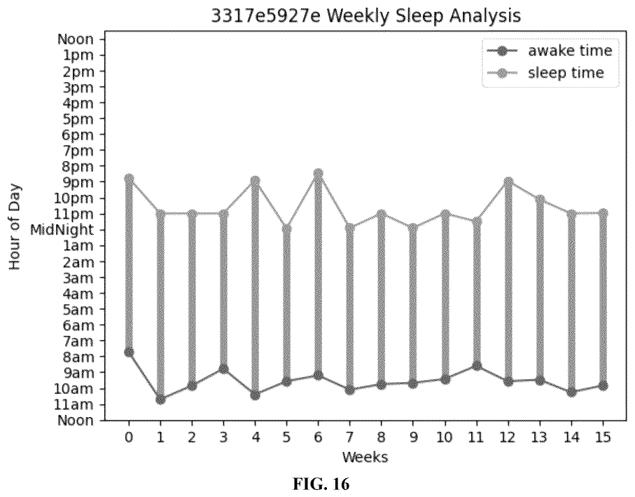

FIG. 16. Sleep pattern week-over-week changes for patient 2.

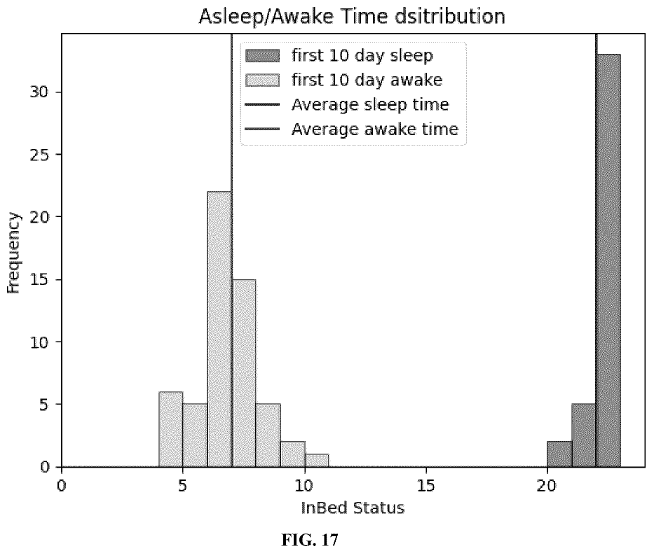

FIG. 17. This diagram illustrates the asleep and awake distribution across all 70+ patients. It is based on weekly aggregation. It tolerates missing data entries for a few days.

The output from system 1 is served as input to system 2: circadian rhythm (day/night boundary) so that the system can segment the temporal data into day segment and night segment. In system 2, the system will conduct personalized distribution analysis and statistical analysis.

### System 2: Personalized Benchmark Establishment

### Biometrics Data Categorization

The inventors categorized the input biometrics dataset by the characteristics of the data stream.

Category 1. The biometrics, such as heart rate, and physical activities show higher frequency throughout the 24 hours of the day. Also, such biometrics exhibit distinct patterns during day vs. night. To conduct granular assessment, the model of the present invention analyzes the data in segmented timespan.

Category 2. For the biometrics that have sparse data points and are worthwhile to generate the pattern in the continuous 24-hour cycle, the model of the present invention analyzes the data in a full circadian cycle. Such biometric features include, but not limited to, heart rate variability, resting heart rate, and oxygen saturation.

Category 3. The extreme cases of sparse data points are the measurements that require patients' manual work: once per day blood pressure measurement (systolic and diastolic) via user input via app, and ECG via manual trigger on wearable devices, such as wearable device.

### Preparing ML Model Input Features.

Step 1: Temporal Data Segmentation. The inventors used the day and night boundary derived from system 1 above (circadian rhythm) to segment the biometrics and physical activities temporal data stream into Day Temporal Data and Night Temporal Data.

Step 2: Personalized Benchmark Threshold Establishment. Based on the segmented temporal data, the system applies distribution analysis to establish the personalized upper threshold and lower threshold for a given subject and given biometrics feature.

For category 1 biometrics/PA: Personal Day Upper Threshold, Personal Day Lower Threshold, Personal Night Upper Threshold, and/or Personal Night Lower Threshold.

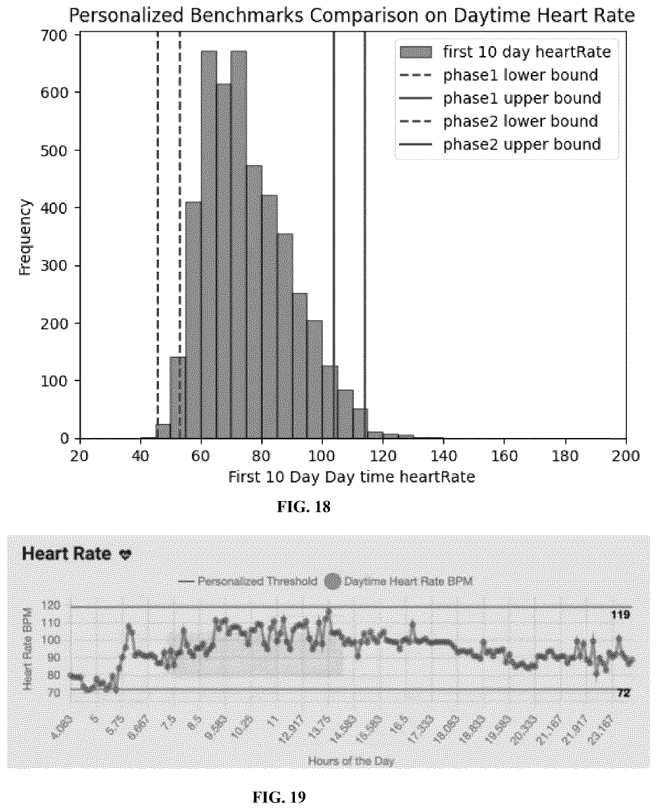

FIG. 18. Heart rate distribution for patient d0eabf86fa from February 18 to February 28, first 10 days into the trial. The diagram illustrates a highly skewed distribution.

The following diagram illustrates the established personalized heart rate benchmarks. The upper benchmark is set at 119, in contrast to the clinical standard of 120, while the lower benchmark is established at 72, diverging from the clinical norm of 60.

FIG. 19. Sample heart rate personalized benchmark. X-axis represents time of the day. Y-axis represents heart rate measurements. The personalized upper benchmark is 119 and lower benchmark is 72.

For category 2 biometrics: Personal Full Circadian Cycle Upper Threshold and Personal Full Circadian Cycle Lower Threshold.

For category 3 biometrics: Preserve the raw data as ML model input features.

Step 3: Daily Personalized Outliers Risk Analysis. Based on the established thresholds, the inventors illustrate the outliers on both the daily risk report as well as the long-term trend report: Personal Daily Day Upper Outliers, Personal Daily Day Lower Outliers, Personal Daily Night Upper Outliers, and Personal Daily Night Lower Outliers.

With the upper and lower threshold establishment, the system of the present invention computes the outliers count and percentage and uses them as part of the ML input features.

Following are the blood oxygen saturation graphs.

FIG. 20. Daily Blood Oxygen Saturation graphs with personal benchmark and outliers colored in red. The personalized upper benchmark is 100 and lower benchmark is 88, whereas the clinical threshold is 100 and 93. Following is the long-term Blood Oxygen Saturation outlier graph.

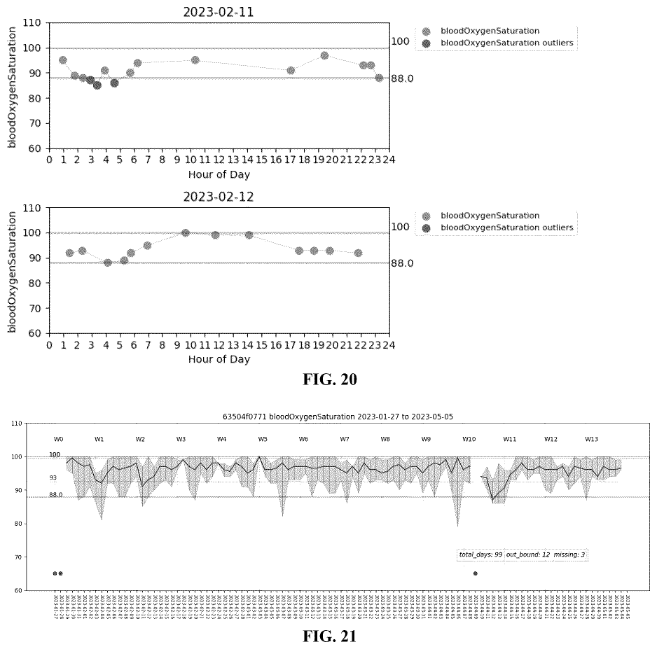

FIG. 21. Long-term Blood Oxygen Saturation graph. The purple lines represent the personalized benchmarks and the green lines represent the clinical thresholds.

Following is the long-term Blood Oxygen Saturation outlier count report.

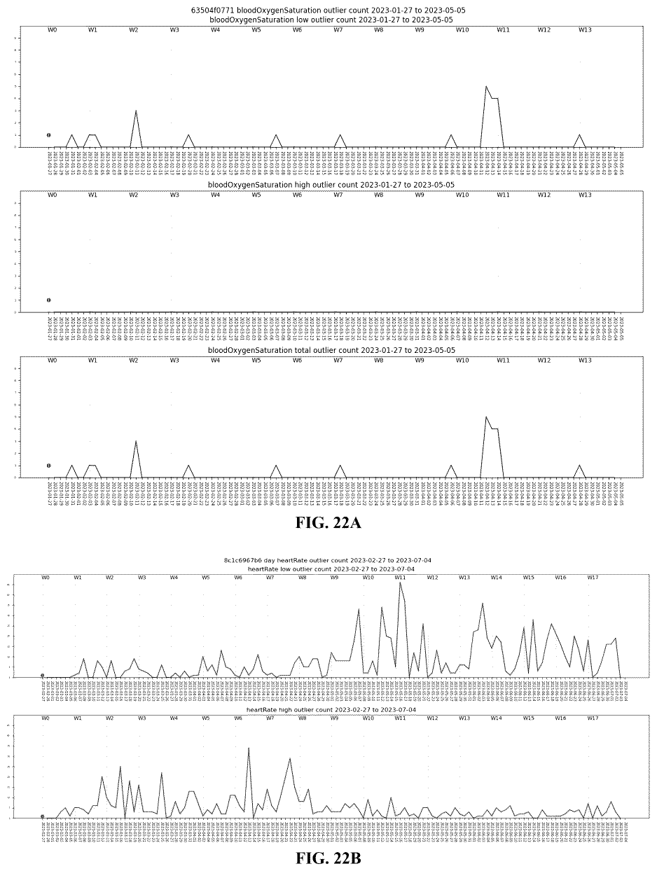

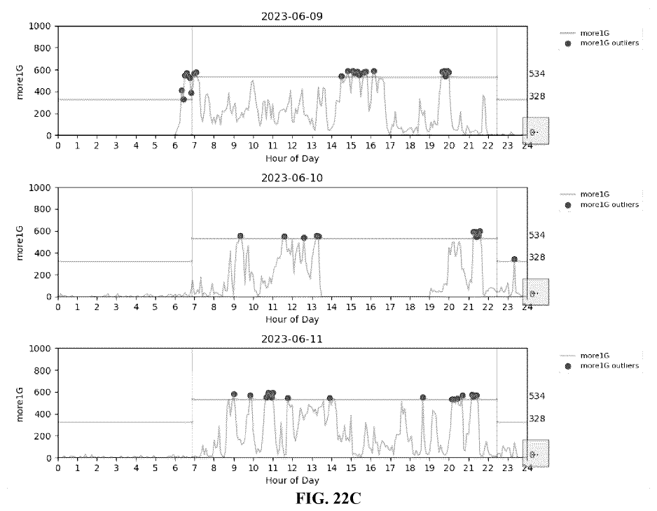

FIG. 22A to 22C shows a long-term blood oxygen saturation outlier report, a daily outlier report, and an hourly outlier report. FIG. 22A the x-axis represents each day, y-axis represents outlier count for the given day. For this specific example, it shows the data points for patient ID 63504f0771 for the clinical trial duration. FIG. 22B shows daily outlier count of the heart rate incidents (low outliers and high outliers) based on personalized benchmark thresholds for daytime segment and nighttime segment. FIG. 22C shows hourly outlier count of the physical activities (>=1 gravity force) based on personalized benchmark thresholds for daytime segment and nighttime segment.

Step 4: Daily Biometrics Statistics Analysis. Based on the segmented temporal data for each biometrics feature, the system of us the present invention computes personalized daily statistical information (min, max, mean, median). Then, the inventors used these results as part of the ML input features.

For category 1 biometrics/PA: Personal Day/Night Min/Max/Mean/Median.

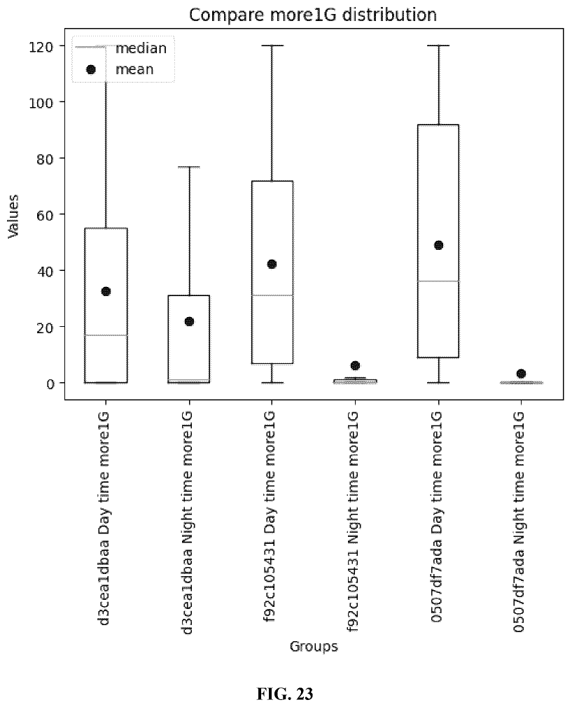

FIG. 23. Personalized Day/Night More 1G distribution over 3 patients (d3cea1dbaa, f92c105431, 0507df7ada).

For category 2 biometrics: Personal Full Circadian Cycle Min/Max/Mean/Median.

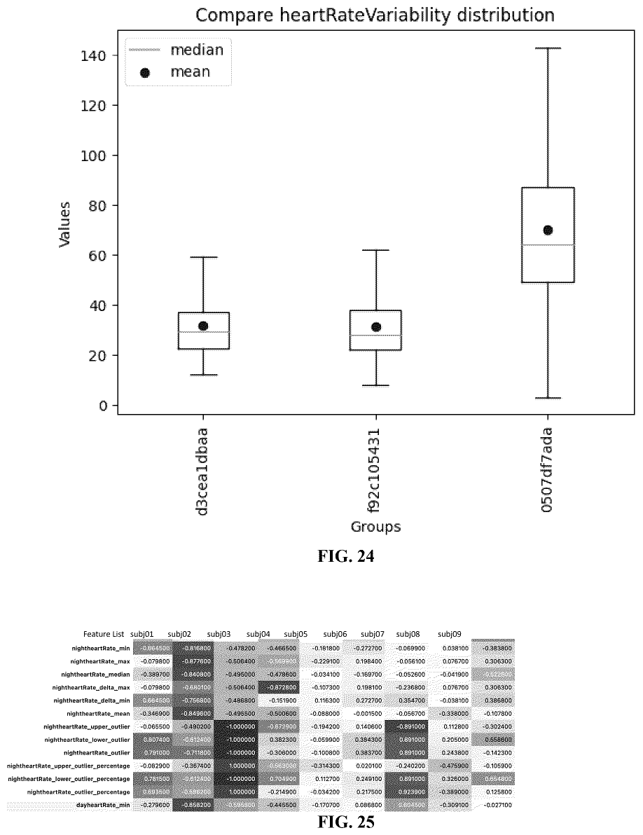

FIG. 24. Heart rate variability distribution for 3 patients (d3cea1dbaa, f92c105431, 0507df7ada). The heart rate variability is one of the premium indicative features for some of the patients, but not for others. The system of the present invention therefore developed the capability to dynamically select the risk indicative features on a patient-by-patient basis.

### Innovation in the AiCare Multimodal AI Framework.

The Innovation in the AiCare multimodal AI framework is as follows. First, one or more multimodal analyses on diversified feature domain, feature categorization and normalization. Second, personalization based on benchmark establishment with biomarker data gathered during one or more time periods, e.g., 0.1, 0.2, 0.3, 0.4, 0.5, 0.6, 0.7, 0.8, 0.9, 1, 2, 3, 4, 5, 6, 7, 8, 9, 10 days, 1, 2, 3, 4 weeks, 1, 2, 3, 4, 5, 6 or more months. Third, additional features are derived based on the personal benchmarks as well as circadian rhythms. The present inventors developed the additional features: personalized benchmark-based outliers, statistic-based features. (See also “Development of the Model Input Features” hereinbelow for details). Fourth, a comprehensive feature domain development (e.g., 10, 20, 25, 30, 40, 50, 60, 70, 75, 80, 90, 100, 110, 125, 130, 140, 150, 175, 200, 250+ features) using the personalized biomarkers. The comprehensive feature domain development includes one or more of the following: (1) The current AI pipeline handles temporal data: establish the benchmark and then monitor/alert the ongoing health conditions based on the comparison with the benchmarks. The genetic markers are not temporal data. The inventors do not analyze genetic markers in the same way as heart rate, etc. The genetic markers are very beneficial in setting up the subjects' cohorts, e.g. subjects sharing the similar gene characteristics. (2) Time (interval-based) series analysis in feature development, use correlation to summarize the change in feature over time progression and use the changes over time as model inputs. (3) Finally, custom cost functions are established.

### Data Preprocessing and Normalization

First, the noise was removed from the data. This included an enhanced solution adoption, viz., addressing outliers such as occasional missing wearable data, the system strengthens the solution adoption. Next, the data intervals were normalized. An example of normalization is, e.g., analyzing the data plot as described hereinbelow. Briefly, physical activity measurements were used as the input feature for training the ML model. The inventors used sleep status from a wearable device as a benchmark label to evaluate the model accuracy. Sleep status labels are categorical values. The inventors used the categorical values “asleep” and “inbed” as training labels. The analysis of the different categories of data is explained next.

For data in category 1, the normalization interval is 1, 60, 300, 3,600 seconds depending on the use cases. Table 5 demonstrates the heart rate raw data. As we can see, the data points are dense (multiple data points within a 5-minute span), therefore, with is then normalized into a fixed time interval.

TABLE 5
Heart rate raw data.

| createLocalTime | heartRate |
|---|---|
| 12:34:13.781 | 73 |
| 12:41:10.784 | 65 |
| 12:42:56.284 | 69 |
| 12:46:20.284 | 62 |
| 12:50:40.159 | 67 |
| 12:54:30.410 | 73 |
| 12:56:58.285 | 76 |

For data in category 2 and 3, the time interval presented in the raw data is inherited, which means that the system does not normalize the data to a unified interval. The following table demonstrates the heart rate variability raw data. In this example, the data points are sparse (4 data points within a 12-hour's time span), therefore, we do not normalize them into a 5-minute interval. These raw data are used as is shown in Table 6.

TABLE 6
Raw data.

| createLocalTime | heartRateVariability |
|---|---|
| 12:47:17.762 | 30 |
| 15:52:01.218 | 25 |
| 20:23:35.447 | 48 |
| 23:37:33.335 | 18 |

### Development of the Model Input Features.

Next, the inventors developed a comprehensive feature list based on claim 1 (circadian rhythm identification) and 2 (personalized benchmark establishment). The inventors developed personalized benchmark-based outliers, statistic-based features:

Examples of outlier-based features are as follows: Heart rate outliers: (1) Day upper outliers: the heart rate data points higher than the established individual's day upper threshold; (2) Day lower outliers: the heart rate data points lower than the established individual's day lower threshold; (3) Night upper outliers: the heart rate data points higher than the established individual's night upper threshold; and/or (4) Night lower outliers: the heart rate data points lower than the established individual's night lower threshold.

Examples of physical activities outlier-based features are as follows: (1) Day 1G upper outliers; (2) Day 1G lower outliers; (3) Night 1G upper outliers; (4) Night 1G lower outliers; and/or (5) Above logic is applied to 2G and 3G as well

Additional outlier-based features are derived from other biomarkers as well, e.g. heart rate variability, resting heart rate, blood oxygen saturation. For example, for blood oxygen saturation, we only need lower outliers based on lower benchmarks. This is because the higher oxygen saturation is not a risk factor. Examples include statistic-base features, e.g., Statistics: min, mean, median, max, and/or sum.

TABLE 7
Examples of samples

nightheartRate_min
nightheartRate_max
nightheartRate_median
nightheartRate_delta_max
nightheartRate_delta_min
nightheartRate_mean
nightheartRate_upper_outlier
nightheartRate_lower_outlier
dayheartRate_min
dayheartRate_max
dayheartRate_median
dayheartRate_delta_max
dayheartRate_delta_min
dayheartRate_mean
dayheartRate_upper_outlier
dayheartRate_lower_outlier

### Iteration of the Models.

The feature values are streamed into the multimodal AI framework. The following are the iteration of the models. (1) P-Value based weight; (2) Z-Score based weight; and/or (3) Multimodal AiCare Framework.

Terminologies. As used herein, the term “Subjects Cohort” refers to those subjects that share the similarities are put into the same group (a.k.a. cohort). For example, subjects who share the same genetic characteristics, or share the same disease symptoms, or share the same health condition progression, or share the same demographic definition.

As used herein, the term “Correlation value ([−1, 1])” represents the correlation between the input feature and time progression (value>0: direct correlation, value<0: inverse correlation).

As used herein, the term “P-value” refers to the ordinary meaning of the statistical determination that the probability of obtaining the observed results, assuming that the null hypothesis is true. Generally, the lower the p-value, the greater the statistical significance of the observed difference. A p-value of 0.05 or lower is generally considered statistically significant. For each feature, perform one sample t test against 0 (no change in this feature over time progression) to get statistical significance of the correlation.

FIG. 25 shows the correlation value of different subjects of different features. Each column represents a different subjectID, each row represents an input feature.

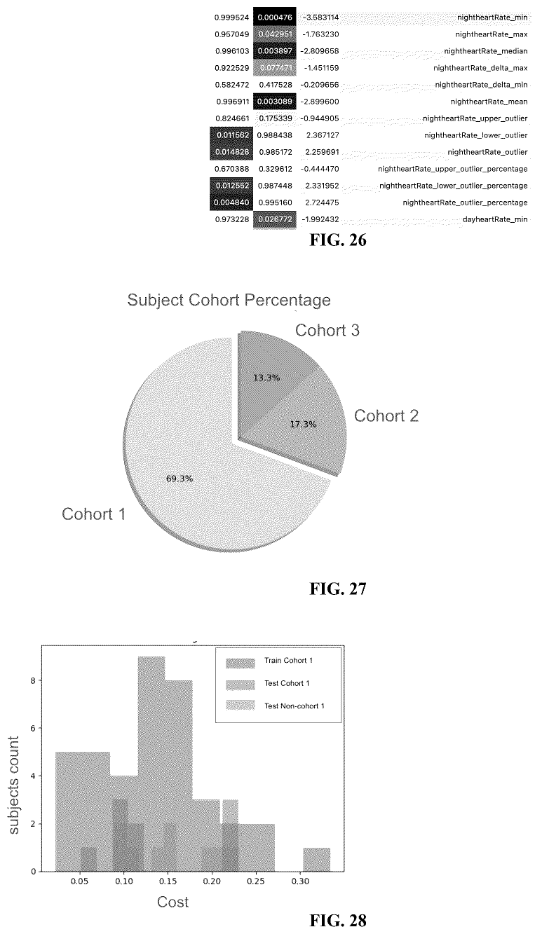

FIG. 26 shows the results from one sample t test over the correlation values for a subjects' cohort (subset of the subjects under study). Each column represents t test statistic result in different directions (feature value increasing or decreasing over time). Each row represents an input feature (label on the right).

### Subjects Cohort Categorization

FIG. 27 is a pie chart that summarized the sample subjects cohorts. In the above diagram, the overall study population is categorized into three cohorts: 13.3%, 17.3% and 69.3% respectively. The subjects in different cohorts demonstrate different characteristics in the study.

### Risk Prediction Models.

First, the inventors determined the results using a P-Value based model. The following parameters were used. Weight: P value and z score calculation: (1) Use correlation value from all subjects in cohort 1; (2) For each feature, calculate the statistical significance of their correlation value (correlation value 0 represents no change in this feature over time progression); and (3) Get the p value and z score of the t test.

Next, the inventors determined the cost function. For each sample: Cost=summation of [(signal value 0 or 1)×(feature correlation deviation from subject cohort mean)×weight assigned to the feature]. For each feature of each subject there exist 3 conditions: (1) Signal value 0 is assigned to the feature with the correlation value that is more significant than the subject cohort's mean; (2) Otherwise, signal value 1 is assigned to the feature, including correlation value inverse with or less significant than the subject cohort's mean; and (3) weight of feature: Weight of feature represents the degree of the statistical significance of the correlation value away from 0.

FIG. 28 is a diagram that illustrates that cohort 1 is not clearly separated from the total subject population. It shows the distribution of the cost value for the p-value based model. The use of the cost function was to distinguish/differentiate subjects belonging to cohort 1 from the rest of the subjects. The process is that the cost was first calculated on a subset of cohort 1 (blue: Train Cohort 1), then the cost function was applied to the remaining test subjects. X-axis represents the cost value. This diagram shows that this cost function cannot effectively separate cohort 1 from the rest. As we can see, among test subjects, cohort 1 (green: Test Cohort 1) and non-cohort 1 (orange: Test Non-cohort 1) were mixed.

Legend Definition: Train cohort 1: cohort 1 subjects' population in the training dataset. Test cohort 1: cohort 1 subjects' population in the test dataset. Test non-cohort 1: those subjects that do not belong to cohort 1 in the test dataset.

TABLE 8
P-value based model performance when conducting cohort 1
subjects' prediction: accuracy, recall, precision for
cohort 1 prediction. The cohort 1 subjects can represent
improving patients, worsening patients, users with risky
health conditions, or users with health condition changes.
Single Model

| | |
|---|---|
| accuracy | 0.667 |
| recall | 0.444 |

| | | |
|---|---|---|
| precision | 0.8 | Prediction |

| | | cohort 1 | non-cohort 1 |
|---|---|---|---|
| Label | cohort 1 | 4 | 5 |
| | non-cohort 1 | 1 | 8 |

Ensembled Model

| | |
|---|---|
| accuracy | 0.556 |
| recall | 0.667 |

| | | |
|---|---|---|
| precision | 0.545 | Prediction |

| | | cohort 1 | non-cohort 1 |
|---|---|---|---|
| Label | cohort 1 | 6 | 2 |
| | non-cohort 1 | 5 | 4 |

Though the p-value based cost function reveals the difference in importance among features, it is a “e-based log” based transformation based on difference of standard deviation, the degree of the difference in p-value does not represent the degree of the disparity among importance of features. Therefore, the following model was developed.

### Z-Score Based Model.

As with the previous example, the inventors next used a Z-Score based model with the following parameters. Weight: P value and z score calculation: (1) Use correlation value from all subjects in cohort 1; (2) For each feature, calculate the statistical significance of their correlation value (correlation value 0 represents no change in this feature over time progression); and (3) Get the p value and z score of the t test.

The inventors also determined the cost function as follows. For each sample: Cost=summation of [(signal value 0 or 1)×(feature correlation deviation from subject cohort mean)×weight assigned to the feature]. For each feature of each subject there exist 3 conditions: (1) Signal value 0 is assigned to the feature with the correlation value that is more significant than the subject cohort's mean. (2) Otherwise, signal value 1 is assigned to the feature, including correlation value inverse with or less significant than the subject cohort's mean. (3) Weight of feature: (a) Weight of feature represents the degree of the statistical significance of the correlation value away from 0. (b) Z scores weights offer a more representative measure of the difference in importance among features. Comparatively, P values serve as a “log” of sorts, derived from Z scores. Consequently, in practice, if the z score weights for features A and B are 1 and 2 respectively, the corresponding P value weights would be approximately 0.8 and 0.9. This illustrates that the relative disparity in importance between feature A and B is better represented with z scores

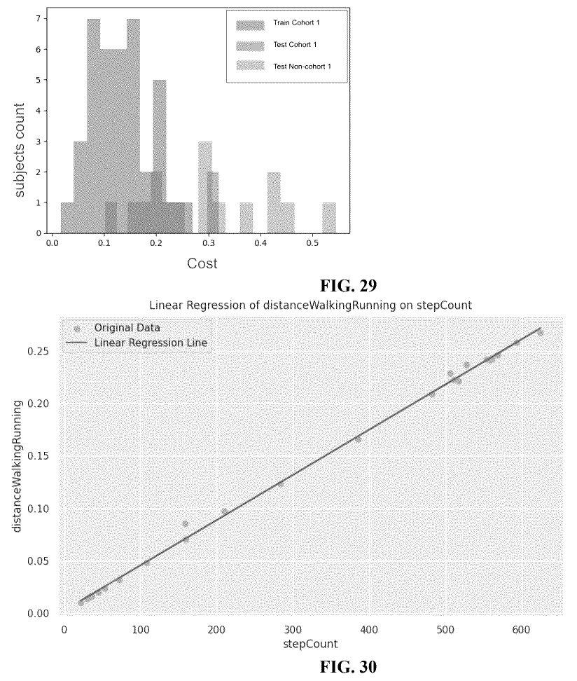

FIG. 29 show the distribution of cost value for the z-score based model. Compared with the p-value based model, this model improves on the separation of cohort 1 from the total subject population. Train cohort 1: cohort 1 subjects' population in the training dataset. Test cohort 1: cohort 1 subjects' population in the test dataset. Test non-cohort 1: those subjects that do not belong to cohort 1 in the test dataset.

Table 9 shows the results from using a Z-score based model performance when conducting cohort 1 subjects' prediction: accuracy, recall, precision for cohort 1 prediction. It shows improvement from P-value based model. The cohort 1 subjects can represent improving patients, worsening patients, users with risky health conditions, or users with health condition changes.

TABLE 9
Z-score based model performance
Single Model

| | |
|---|---|
| accuracy | 0.667 |
| recall | 0.333 |

| | | |
|---|---|---|
| precision | 1 | Prediction |

| | | cohort 1 | non-cohort 1 |
|---|---|---|---|
| Label | cohort 1 | 3 | 6 |
| | non-cohort 1 | 0 | 9 |

Ensembled Model

| | |
|---|---|
| accuracy | 0.889 |
| recall | 0.778 |

| | | |
|---|---|---|
| precision | 1 | Prediction |

| | | cohort 1 | non-cohort 1 |
|---|---|---|---|
| Label | cohort 1 | 7 | 2 |
| | non-cohort 1 | 0 | 9 |

Based on the results above, the inventor next developed a multimodal AI framework.

Table 10 summarizes the multimodal AI framework performance when conducting cohort 1 subjects' prediction: accuracy, recall, precision. The model performance is improved significantly compared with the z-score based model and p-value based model. The cohort 1 subjects can represent improving patients, worsening patients, users with risky health conditions, or users with health condition changes.

TABLE 10
Multimodal AI framework performance.
Single Model

| | |
|---|---|
| accuracy | 0.937 |
| recall | 0.958 |

| | | |
|---|---|---|
| precision | 0.958 | Prediction |

| | | cohort 1 | non-cohort 1 |
|---|---|---|---|
| Label | cohort 1 | 46 | 2 |
| | non-cohort 1 | 2 | 13 |

Ensembled Model

| | |
|---|---|
| accuracy | 0.937 |
| recall | 0.923 |

| | | |
|---|---|---|
| precision | 1.000 | Prediction |

| | | cohort 1 | non-cohort 1 |
|---|---|---|---|
| Label | cohort 1 | 48 | 0 |
| | non-cohort 1 | 4 | 11 |

### Model to Fit Healthcare Use Case.

Due to the characteristics of the healthcare domain and the strengths of the different AI/ML models, the multimodal AI framework and ensemble models were used a model to fit healthcare use case.

The inventors recognized the uniqueness of the healthcare domain and datasets, including one or more of the following features of the datasets: (1) Explainability and clarity of the decisions in the model; (2) Complexity and non-linear separation in the feature domains; (3) Elasticity of the size of the dataset. This means the size of the datasets can be significantly different, ranging from a few subjects for a few weeks, to hundreds of subjects for years of longitudinal study; (4) Diversity of the data types (categorical discrete values vs. numerical continuous values); and/or (5) Unbalanced dataset with majority of the data points representing the healthy subjects or non-risky conditions of the subjects.

### Feature Selection Process.

Following is the feature engineering process. System deploys three approaches: (1) noise reduction; (2) shares features that trend within cohort; and (3) high indicative features based on standard deviation. Each is explained in detail hereinbelow.

### Approach 1: Noise Reduction.

The purpose of this process was to reduce the noise from the non-essential features. Before streaming the data into the model, feature selection is performed. Cross features linear regression analysis was used to conduct dimensionality reduction.

FIG. 30 shows a linear regression analysis between distanceWalkingRunning and stepCount. System reduces feature distanceWalkingRunning and keeps stepCount because they're highly correlated.

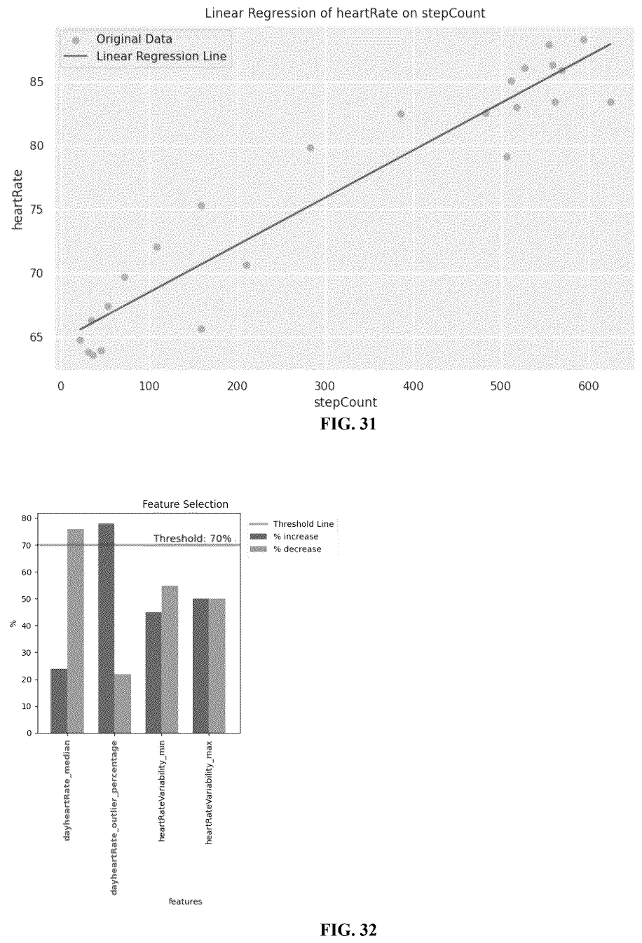

FIG. 31 shows a linear regression analysis between heartRate and stepCount. System keeps both of these features because their correlation is not significantly high.

Approach 2: Share the Feature Trend within Cohort

System evaluates each of the features in the selection process. If a certain percent, e.g. 70%, 75%, 80%, of the subjects within a cohort show the same trend direction on a given feature (increasing/decreasing over time), then the system will automatically select this feature. Otherwise, the feature will be dropped.

FIG. 32 shows the sample feature selection. The features will be considered in the model if more than a given percentage of subjects in the cohort (70% in this example) share the trend in the same direction. In this illustration, selected features are dayheartRate_median and dayheartRate_outlier_percentage.

### Approach 3: High Indicative Features Based on Standard Deviation

Based on the correlation score for a given feature and a given subject, the system selects high indicative features: correlation score's standard deviation>threshold of 0.3, 0.4, 0.5. Please refer to Table 11: Correlation Value Standard Deviation, for details.

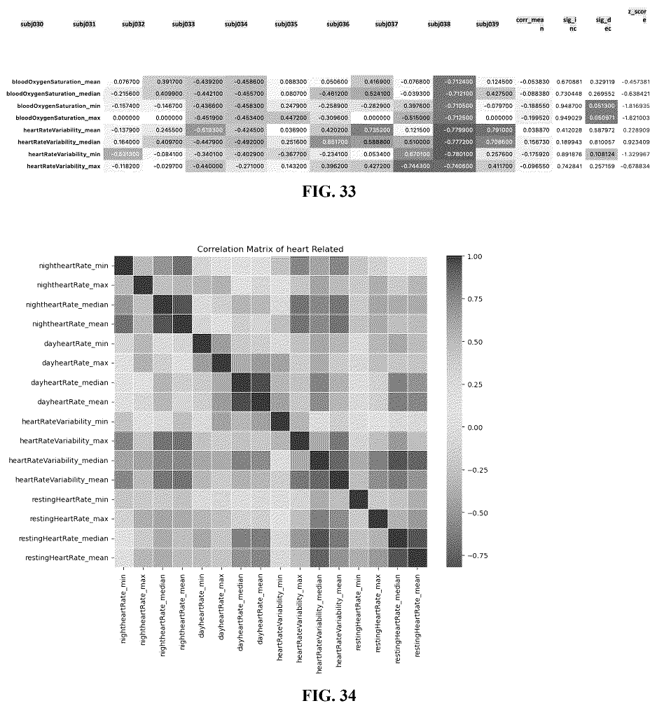

FIG. 33 shows a correlation scores for a given feature and a given subject. Each column represents one subject. Each row represents one feature.

### Biomarker Cluster Analysis

Next, the inventors conducted a tTest for feature correlation normality: Shapiro-Wilk test: en.wikipedia.org/wiki/Shapiro-Wilk_test

$$W = \frac{{( {\sum }_{i = 1}^{n} {a}_{i} {x}_{( i )} )}^{2}}{{\sum }_{i = 1}^{n} {( {x}_{i} - \overline{x} )}^{2}} ,$$

### Correlation Between Features:

Correlations are calculated between each pair of features. The value for each feature are first aggregated by week. Then, the correlation is calculated between the weekly value array of the features.

FIG. 34 shows the correlation between heart-related features. Each square represents the value of correlation (with deep red indicating values close to 1 and deep blue indicating values close to −1). Features belonging to the same biomarkers usually show stronger correlation (groups of red squares near the diagonal). Some relationships are also observed between features of different biomarkers. For example, the mean, max, and median of heart rate variability are strongly negatively correlated with night heart rate-related features and resting heart rate-related features (blue squares).

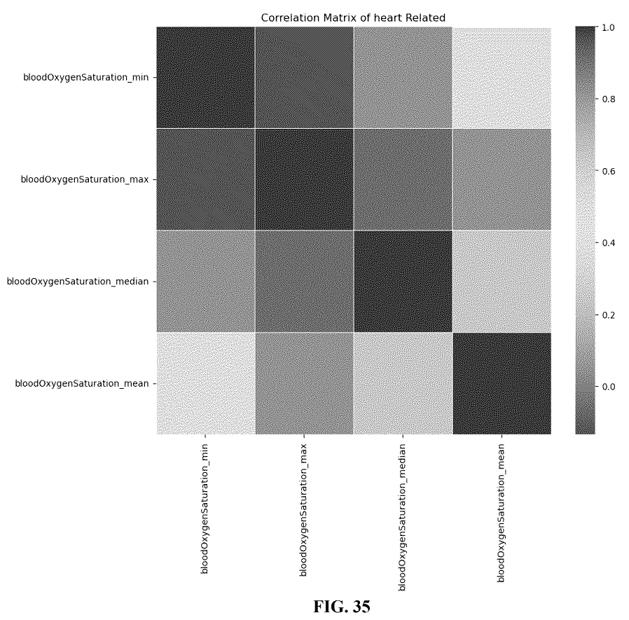

FIG. 35 shows the correlation heat map between blood Oxygen related features

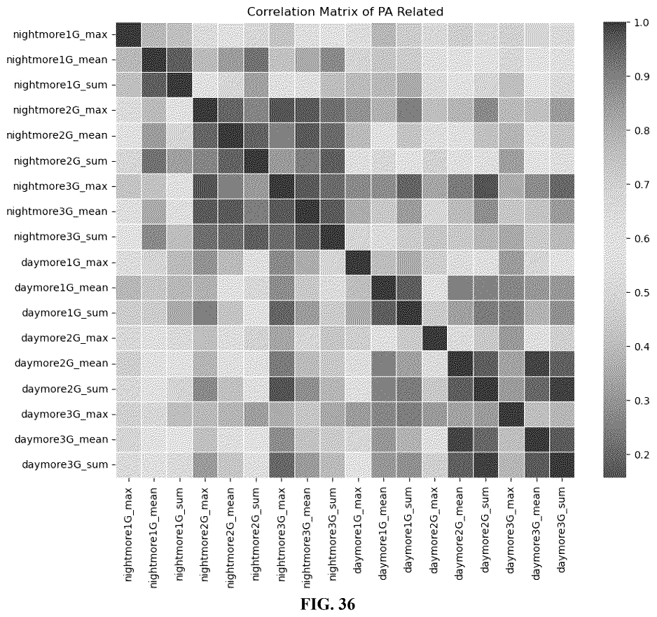

FIG. 36 shows the correlation heatmap among physical activity (more1G, more2G, more3G) related features. Features for night physical activities are strongly positively correlated with each other. Night more1G features are weakly positively correlated with day physical activities. However, night more2G and night more3G features are negatively correlated with day physical activity features.

### Sample Model Configuration

Some of the hyperparameters are adjusted to fit different business use cases. Ensemble Model: To train each single model in the ensemble model, a subset of features are used for training. The number of features in the subset is the square root of the total number of features available. Then, the top 5 most important features in each subset are used to draw the decision boundary between cohorts.

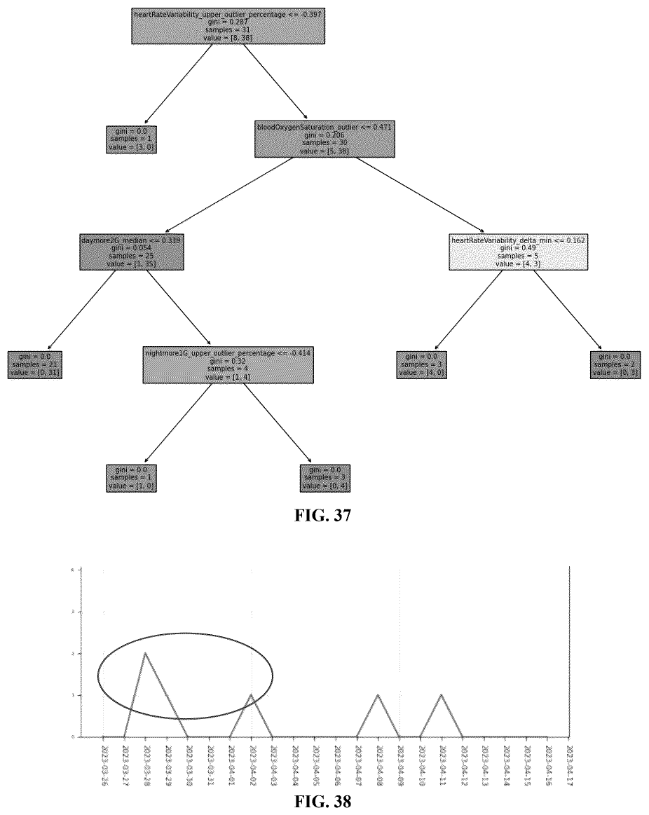

FIG. 37 is a flowchart that illustrates the explainability of the ML model which is a requirement or strongly preferred feature in the healthcare domain. The higher the feature in the decision process, in general, it carries more weight. The colors indicate whether the majority of samples in the subgroup at this stage of the tree belong to Group 1 (blue) or Group 2 (orange). The higher intensity of the color indicates a greater proportion of the subgroup belonging to one group compared to the other. A more intense color signifies a higher purity of the node, meaning most samples in that node belong to a single group, while a lighter color indicates a more mixed distribution of samples from both groups.

TABLE 11
Correlation Value Standard Deviation

| | |
|---|---|
| nightmore3G_median | 0.1505796592 |
| daymore1G_min | 0.1842703503 |
| daymore2G_min | 0.1842703503 |
| daymore3G_min | 0.1842703503 |
| nightmore1G_min | 0.1935524153 |
| nightmore1G_delta_min | 0.1935524153 |
| nightmore1G_lower_outlier | 0.1935524153 |
| nightmore1G_lower_outlier_percentage | 0.1935524153 |
| nightmore2G_min | 0.1935524153 |
| nightmore2G_delta_min | 0.1935524153 |
| nightmore2G_lower_outlier | 0.1935524153 |
| nightmore2G_lower_outlier_percentage | 0.1935524153 |
| nightmore3G_min | 0.1935524153 |
| nightmore3G_delta_min | 0.1935524153 |
| nightmore3G_lower_outlier | 0.1935524153 |
| nightmore3G_lower_outlier_percentage | 0.1935524153 |
| daymore1G_delta_min | 0.1935524153 |
| daymore1G_lower_outlier | 0.1935524153 |
| daymore1G_lower_outlier_percentage | 0.1935524153 |
| daymore2G_delta_min | 0.1935524153 |
| daymore2G_lower_outlier | 0.1935524153 |
| daymore2G_lower_outlier_percentage | 0.1935524153 |
| daymore3G_delta_min | 0.1935524153 |
| daymore3G_lower_outlier | 0.1935524153 |
| daymore3G_lower_outlier_percentage | 0.1935524153 |
| bloodOxygenSaturation_delta_max | 0.1935524153 |
| bloodOxygenSaturation_upper_outlier | 0.1935524153 |
| bloodOxygenSaturation_upper_outlier_percentage | 0.1935524153 |
| nightmore2G_median | 0.2465857767 |
| daymore3G_median | 0.2727207941 |
| bloodOxygenSaturation_max | 0.2817229655 |
| bloodOxygenSaturation_lower_outlier | 0.3384450439 |
| bloodOxygenSaturation_outlier | 0.3384450439 |
| dayheartRate_delta_min | 0.3400342731 |
| bloodOxygenSaturation_delta_min | 0.3572999158 |
| heartRateVariability_upper_outlier | 0.3583083781 |
| dayheartRate_min | 0.3611035427 |
| nightmore1G_median | 0.3657745475 |
| heartRateVariability_lower_outlier_percentage | 0.3681866848 |
| bloodOxygenSaturation_min | 0.3701068135 |
| heartRateVariability_outlier | 0.3705119798 |
| dayheartRate_max | 0.3714611884 |
| nightmore1G_upper_outlier_percentage | 0.3715210305 |
| nightmore1G_outlier_percentage | 0.3715210305 |
| heartRateVariability_delta_max | 0.3728066381 |
| nightmore1G_upper_outlier | 0.374075439 |
| nightmore1G_outlier | 0.374075439 |
| dayheartRate_delta_max | 0.3757251018 |
| nightheartRate_min | 0.375999398 |
| restingHeartRate_upper_outlier_percentage | 0.3770445967 |
| heartRateVariability_max | 0.3807715363 |
| heartRateVariability_delta_min | 0.3819111462 |
| nightheartRate_delta_min | 0.3824261145 |
| restingHeartRate_delta_max | 0.3824966351 |
| nightheartRate_mean | 0.3830416858 |
| nightmore2G_delta_max | 0.3842026474 |
| nightheartRate_upper_outlier_percentage | 0.387300862 |
| nightmore2G_max | 0.387355224 |
| dayheartRate_upper_outlier | 0.388430836 |
| nightheartRate_median | 0.3892904433 |
| nightmore1G_delta_max | 0.3896732846 |
| nightmore1G_max | 0.38967551 |
| restingHeartRate_max | 0.3898504106 |
| heartRateVariability_lower_outlier | 0.3902175488 |
| daymore3G_delta_max | 0.3946438523 |
| restingHeartRate_median | 0.3954892127 |
| daymore3G_max | 0.3976312282 |
| dayheartRate_lower_outlier | 0.3979456583 |
| restingHeartRate_upper_outlier | 0.3991593921 |
| nightmore3G_mean | 0.3996248021 |
| daymore2G_delta_max | 0.4013393962 |
| nightheartRate_delta_max | 0.401545683 |
| nightheartRate_lower_outlier | 0.4028577145 |
| daymore2G_max | 0.403124274 |
| restingHeartRate_mean | 0.4065478386 |
| nightmore2G_mean | 0.4088649465 |
| dayheartRate_outlier | 0.4096216581 |
| nightmore3G_delta_max | 0.4131338286 |
| dayheartRate_upper_outlier_percentage | 0.4134026004 |
| nightmore3G_max | 0.4159987165 |
| nightheartRate_max | 0.4161501848 |
| daymore1G_delta_max | 0.4179816947 |
| nightmore2G_upper_outlier_percentage | 0.4180955704 |
| nightmore2G_outlier_percentage | 0.4180955704 |
| nightmore1G_sum | 0.4203168879 |
| nightmore3G_sum | 0.4228068989 |
| nightmore2G_upper_outlier | 0.4229747515 |
| nightmore2G_outlier | 0.4229747515 |
| nightmore1G_mean | 0.4241848856 |
| nightmore2G_sum | 0.4253986436 |
| heartRateVariability_upper_outlier_percentage | 0.4284074339 |
| nightheartRate_lower_outlier_percentage | 0.4293221455 |
| nightheartRate_outlier | 0.4323057967 |
| daymore2G_median | 0.4323471511 |
| nightheartRate_upper_outlier | 0.4343188736 |
| nightmore3G_upper_outlier_percentage | 0.4384181864 |
| nightmore3G_outlier_percentage | 0.4384181864 |
| daymore1G_upper_outlier | 0.4489268309 |
| daymore1G_outlier | 0.4489268309 |
| nightmore3G_upper_outlier | 0.4494682719 |
| nightmore3G_outlier | 0.4494682719 |
| restingHeartRate_lower_outlier | 0.4507176148 |
| heartRateVariability_min | 0.4529925811 |
| restingHeartRate_min | 0.4547074392 |
| restingHeartRate_lower_outlier_percentage | 0.4549195406 |
| bloodOxygenSaturation_median | 0.456503924 |
| heartRateVariability_outlier_percentage | 0.4582498918 |
| daymore1G_max | 0.4596755602 |
| daymore1G_sum | 0.4690143479 |
| daymore1G_median | 0.4727949953 |
| restingHeartRate_outlier | 0.4749803304 |
| dayheartRate_median | 0.4750920869 |
| daymore3G_upper_outlier | 0.4765911436 |
| daymore3G_outlier | 0.4765911436 |
| nightheartRate_outlier_percentage | 0.4770158327 |
| daymore3G_sum | 0.4806233809 |
| bloodOxygenSaturation_lower_outlier_percentage | 0.480967747 |
| bloodOxygenSaturation_outlier_percentage | 0.480967747 |
| dayheartRate_mean | 0.4845663559 |
| restingHeartRate_delta_min | 0.4896596311 |
| heartRateVariability_mean | 0.4902218319 |
| heartRateVariability_median | 0.4906448096 |
| daymore1G_upper_outlier_percentage | 0.496926857 |
| daymore1G_outlier_percentage | 0.496926857 |
| daymore3G_upper_outlier_percentage | 0.4980203233 |
| daymore3G_outlier_percentage | 0.4980203233 |
| daymore2G_sum | 0.4985512007 |
| daymore3G_mean | 0.5043625321 |
| daymore2G_upper_outlier | 0.5050483541 |
| daymore2G_outlier | 0.5050483541 |
| daymore1G_mean | 0.5129038564 |
| dayheartRate_lower_outlier_percentage | 0.527755942 |
| daymore2G_mean | 0.5316953121 |
| daymore2G_upper_outlier_percentage | 0.5324884685 |
| daymore2G_outlier_percentage | 0.5324884685 |
| bloodOxygenSaturation_mean | 0.5369137966 |
| dayheartRate_outlier_percentage | 0.6039275108 |
| restingHeartRate_outlier_percentage | 0.6278946985 |

The following case studies demonstrate how the AiCare methodology was used with patients for both deterioration and recovery situations. Please note that the AiCare system is not a diagnosis system, it's a system to predict the health condition deterioration or recovery changes.

### Case Study 1: Single Patient Risk Prediction

A male patient in the age range 65-75. Symptom and diagnosis journey: stomachache on day 1, high fever up to 104.7 F on day 3 through day 6. Gastroscopy procedure and abdominal CT were conducted on day 6 and 7 respectively. Patient was hospitalized. Also, E. coli bacteria blood culture was performed. Diagnosed with liver cysts infection. Patient stayed in hospital for 7 days with IV antibiotics. Patient was recovered and discharged.

The patient wore an Apple Watch and was monitored by AiCare risk prediction system before, during and after this liver cysts infection episode. The following diagram shows that the AI system effectively predicted risk on day −4, which was 4 days before the initial symptom (stomachache).

FIG. 38 is a graph that shows the use of the risk score prediction was able to identify an episode 4 days prior the symptoms being reported by the patient, which was then in need a medical intervention, the x-axis represents the day, y-axis represents the risk score. The higher the score, the higher the risk. One or more risk scores can be based on high value predictive nodes. However, a final risk score can also be generated using only the subset of nodes that provide strong predictive signals. Here, node gating can again be automatic, manual, or based on parameters that can be adjusted by a user. Based on the risk score prediction can be based on a node level interpretability. The ML Model can include mechanisms for explaining which nodes contributed most to a given prediction.

### Case Study 2: Single Patient Risk Prediction

A female patient in the age range 45-55. Symptom and diagnosis journey: on day 1, the patient showed no symptoms, but AiCare risk prediction system showed a prior warning and suggested patient to check with COVID-19 home test kit and result showed negative. The AiCare system continued showing risk. On day 3, the patient re-tested and turned positive.

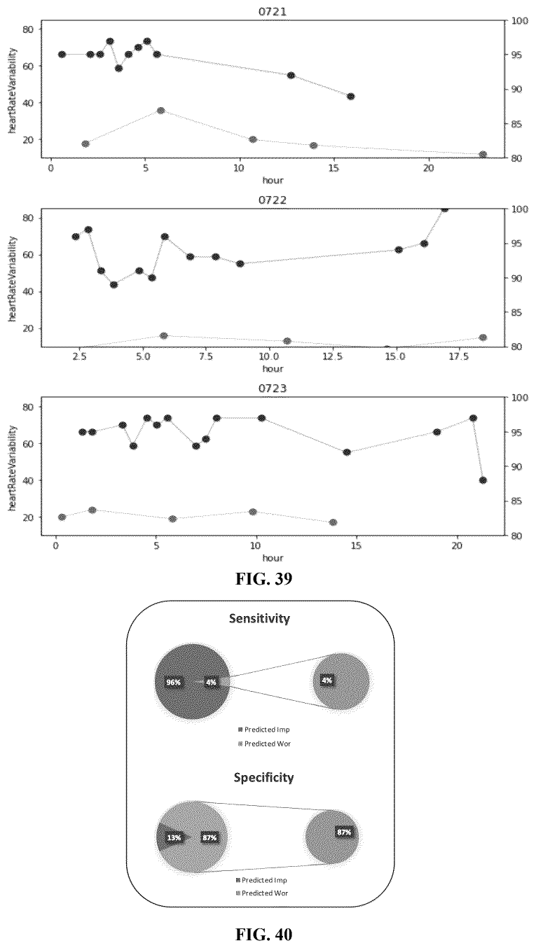

FIG. 39 shows three graphs that include AiCare's risk prediction based on a personalized benchmark prior to diagnosis with COVID. Based on this specific user's condition, the benchmark for heart rate variability was established as >40 for the peak value at night. On day 1, the value dropped below personal benchmark, along with other biomarkers, AiCare system showed risk. On day 3, the user was diagnosed with COVID positive result.

### Case Study 3: Single Patient Recovery Prediction: AI Model Performance Evaluation

This case study presents a holistic AI model performance on recovery prediction. The prediction was performed on individual patients with a total of 90+ long-COVID patients. 70% of the patients were used for training and 30% for testing. Random shuffle and cross-validation were used for testing. AI model performance to predict clinical improvement (based on wearable data): sensitivity: 95.83%, specificity: 86.67%, accuracy: 93.65%.

FIG. 40 shows the results of the AiCare AI model being applied to a total of 90+ long-COVID patients to predict individuals' clinical improvement. The dataset was based on wearable data. Model performance: sensitivity: 95.83% (˜96%), specificity: 86.67% (˜87%), accuracy: 93.65%.

### Case Study 4: Cohort Patients Recovery Prediction: Symptoms Analysis

This is a statistical analysis instead of a single patient analysis. Long COVID, also known as post-acute sequelae of SARS-CoV-2 infection (PASC), refers to a range of symptoms that persist for weeks, months, or even longer after recovering from the acute phase of COVID-19. It can affect multiple organ systems and significantly impact daily life. Symptoms vary across individuals with the leading ones such as brain fog, fatigue, short of breath, stomachache, body ache, cardiovascular symptoms. AiCare's longitudinal study on recovery journeys found that for a significant portion of the population, improvement in brain fog symptoms is often accompanied by a reduction in fatigue. Similarly, patients recovering from symptoms like shortness of breath and stomachache also experience a decrease in fatigue. However, this correlation is not bidirectional-improvement in fatigue symptoms does not necessarily coincide with the recovery of brain fog or other associated symptoms.

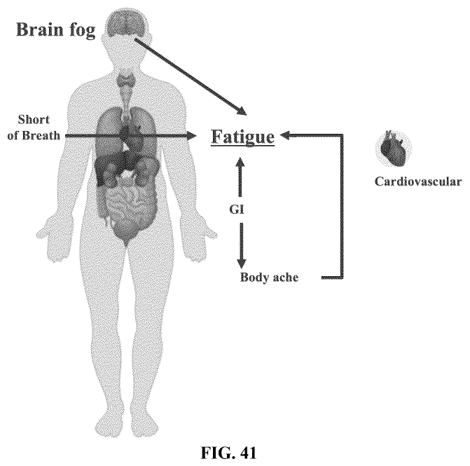

FIG. 41 summarizes 6 major long-COVID symptoms from a multi-patient analysis using the present invention. Each arrow is from a subject cohort experiencing a specific symptom at severe level. When such cohorts recover on this specific symptom, their fatigue is reduced as well. However, for the subject cohort with severe fatigue, improvement in their fatigue does not necessarily lead to a reduction in other symptoms.

The method of the present invention can be used with any type of biological sample as a data source for the machine learning portion, and once the personalized risk assessment is conducted, to treat a disease or condition that is detected by monitoring the biometric data and processing it through the machine learning model of the present invention, whether at an individual level or for groups of individuals. Biometric data from groups of individuals can be processed using the present invention to obtain a baseline, which baseline can then be applied to individual biometric data to determine a personalized risk assessment. The personalized risk assessment is then used to direct treatment of the individual, as the personalized risk assessment provides sufficient details after application of the machine learning model taught herein to identify, in some cases before the individual recognizes symptoms, the changes in biometric data that can be used to direct which types of additional biometric data, biological sample testing, etc., to be used to confirm a diagnosis or determination of a disease or condition, which can then be specifically treated. Non-limiting examples of treatments that can be performed following a change in the personalized risk assessment are summarized as follows.

The methods disclosed herein include administering a therapeutically effective amount of compounds, pharmaceutical compositions, and treatment regimens to a mammal, such as a human. The subject is administered a treatment, after the personalized risk assessment is used to identify a disease, disorder, or condition, and that determination is then used for selecting a treatment for the disease, disorder, or condition, thereby improving patient health and functioning. For example, certain compounds or treatment regimens are used to treat a condition, which is monitored herein to make sure that the patient is improving as measured using the personalized risk determination taught herein. In some embodiments, the compounds are provided directly, or the compounds are used in the manufacture of a medicament to treat the disease, disorder, or condition. In some embodiments, described are methods of administering disclosed compounds to a subject having a condition, such as a disease or disorder, thereby treating the condition.

The compounds or pharmaceutical compositions are administered to a subject by one or more routes of administration, including, e.g., oral, mucosal, rectal, subcutaneous, intravenous, intramuscular, intranasal, inhaled, and transdermal routes. When administered through one or more of such routes, the compound(s) of the disclosure and the disclosed compositions and formulations comprising them are useful in methods for treating a patient in need of such treatment.

As used herein, the phrases “an effective amount” or “a pharmacologically effective amount” refer to an amount of an active agent that is generally non-toxic and sufficient to provide the desired therapeutic effect with performance at a reasonable benefit/risk ratio attending any medical treatment. The effective amount will vary depending upon the subject and the disease condition being treated or health benefit. Factors that can be considered when determining a dosage will be known to those of skill in the art such as, e.g., weight, sex, and age of the subject, severity of the disease condition or degree of health benefit sought, manner of administration, all of which can readily be determined by one of ordinary skill in the art.

As used herein the terms “therapeutic effect” or “therapeutic efficacy” refer to a response in a mammal as measured by the present disclosure, such as a human, after administering the compound(s) and/or regimen(s) that are judged to be desirable and/or beneficial to treat that specific disease, disorder, or condition. Depending on the disease, disorder, or condition to be treated, or improvement in health or functioning sought, and depending on the particular constituent(s) in the compositions under consideration, the therapeutic effect will be readily understood by those of ordinary skill in the art.

The present disclosure provides methods of treating and/or preventing a condition in a mammal, the method comprising administering to the mammal a therapeutically effective and/or prophylactically effective amount of a formulation with one or more active agents. As used herein, the terms “treating” or “treatment” refer to any treatment of a disorder in a mammal, and preferably in a human, and includes causing a desired biological or pharmacological effect as above, as well as any one or more of: (a) preventing a disorder from occurring in a subject who may be predisposed to the disorder but has not yet been diagnosed with it; (b) inhibiting a disorder, i.e. arresting its development; (c) relieving a disorder, i.e., causing regression thereof, (d) protection from or relief of a symptom or pathology caused by or related to a disorder; (e) reduction, decrease, inhibition, amelioration, or prevention of onset, severity, duration, progression, frequency or probability of one or more symptoms or pathologies associated with a disorder; and (f) prevention or inhibition of a worsening or progression of symptoms or pathologies associated with a disorder or comorbid with a disorder. Other such measurements, benefits, and surrogate or clinical endpoints, alone or in combination, will be understood to one of ordinary skill based on the teachings herein and the knowledge in the art.

As used herein, the terms “effective amount,” “therapeutically effective amount,” and/or “pharmacologically effective amount” refer to an amount of an active agent that is generally non-toxic and sufficient to provide a desired therapeutic effect at a reasonable benefit/risk ratio. The skilled artisan will recognize that an effective amount will vary depending upon the subject and the disease condition being treated or health benefit sought, weight, sex, and age of the subject, severity of the disease, condition, or disorder, health benefit sought, and/or dose and route of administration.

For viral infections one of more of the following can be administered to the subject based on the personalized risk assessment: Acyclovir (Zovirax): Treats herpes simplex virus (HSV) and varicella zoster virus (VZV); Amantadine: Treats influenza A virus; Oseltamivir (Tamiflu): Treats influenza; Peramivir: Treats the flu in people 6 months and older; Adefovir: Treats hepatitis B; Ampligen: Treats avian influenza; Ritonavir, atazanavir, and darunavir: Inhibitors of protease; Tenofovir, valganciclovir, and valacyclovir: Inhibitors of viral DNA polymerase; Raltegravir: Inhibitor of integrase, Penciclovir for herpes, or Pleconaril for picornavirus, Paxlovid and/or Lagevrio for coronaviral infections. Over-the-counter medications that can used t to treat the symptoms of viral infections like the common cold and upper respiratory illness: Acetaminophen (Tylenol) for pain and fever; Ibuprofen (Advil) or naproxen (Naprosyn) for pain; Antihistamines for nasal congestion; Oral decongestants for stuffy nose; Guaifenesin for easier nose blowing, and/or Dextromethorphan for suppressing cough. Other antiviral include antisense drugs which use segments of DNA or RNA to block the operation of viral genomes, such as Fomivirsen, a phosphorothioate antisense drug used to treat eye infections in AIDS patients or morpholino oligos, an antisense structural type used to suppress many viral types.

For bacterial infections one of more of the following can be administered to the subject based on the personalized risk assessment: Penicillins that are used to treat a variety of infections, including skin, chest, and urinary tract infections. Examples include penicillin, amoxicillin, and co-amoxiclav. Cephalosporins that are used to treat a wide range of infections, including some more serious infections like sepsis and meningitis. Examples include cefalexin, cefaclor, and cefadroxil. Aminoglycosides that are generally only used in hospitals to treat very serious illnesses like sepsis. Examples include gentamicin and tobramycin. Tetracyclines that are used to treat a wide range of infections, including acne, rosacea, pneumonia, and other respiratory tract infections. Examples include tetracycline, doxycycline, and lymecycline. Macrolides that are used to treat lung and chest infections, and as an alternative to penicillin. Examples include azithromycin, erythromycin, and clarithromycin. Fluoroquinolones that are used to treat a wide range of infections. Examples include ciprofloxacin, levofloxacin, and norfloxacin.

For cardiovascular disease one of more of the following can be administered to the subject based on the personalized risk assessment: Anticoagulants include: Apixaban (Eliquis), Dabigatran (Pradaxa), Edoxaban (Savaysa), Heparin (various), Rivaroxaban (Xarelto), Warfarin (Coumadin), which help prevent blood clots from forming in the blood vessels and are often prescribed to prevent first or recurrent stroke or heart attack. Antiplatelet agents and dual antiplatelet therapy (DAPT) include: Aspirin, Clopidogrel (Plavix), Dipyridamole (Persantine), Prasugrel (Effient), Ticagrelor (Brilinta), which keep blood clots from forming by preventing blood platelets from sticking together. Dual antiplatelet therapy (DAPT) agents include Benazepril (Lotensin), Captopril (Capoten), Enalapril (Vasotec), Fosinopril (Monopril), Lisinopril (Prinivil, Zestril), Moexipril (Univasc), Perindopril (Aceon), Quinapril (Accupril), Ramipril (Altace), Trandolapril (Mavik), which lowers blood pressure by widening blood vessels, thereby reducing the workload of the heart. Angiotensin II receptor blockers (or inhibitors) include: Azilsartan (Edarbi), Candesartan (Atacand), Eprosartan (Teveten), Irbesartan (Avapro), Losartan (Cozaar), Olmesartan (Benicar), Telmisartan (Micardis), Valsartan (Diovan), which prevent angiotensin II from having any effect on the heart and blood vessels. This keeps blood pressure from rising. Angiotensin receptor-neprilysin inhibitors (ARNIs) include: Sacubitril/valsartan (Entresto), which improve artery opening and blood flow, reduce sodium (salt) retention, and decrease strain on the heart. Beta blockers include: Acebutolol (Sectral), Atenolol (Tenormin), Betaxolol (Kerlone), Bisoprolol/hydrochlorothiazide (Ziac), Bisoprolol (Zebeta), Metoprolol (Lopressor, Toprol XL), Nadolol (Corgard), Propranolol (Inderal), Sotalol (Betapace), which slow the heart rate and force of contraction thereby lowering blood pressure, which makes the heartbeat more slowly and with less force. Combined alpha and beta-blockers include: Carvedilol (Coreg, Coreg CR), Labetalol hydrochloride (Normodyne, Trandate). Calcium channel blockers include: Amlodipine (Norvasc), Diltiazem (Cardizem, Tiazac), Felodipine (Plendil), Nifedipine (Adalat, Procardia), Nimodipine (Nimotop), Nisoldipine (Sular), Verapamil (Calan, Verelan), which interrupt the movement of calcium into the cells of the heart and blood vessels. May decrease the heart's pumping strength and relax blood vessels. Cholesterol-lowering medications include: Statins: Atorvastatin (Lipitor), Fluvastatin (Lescol), Lovastatin (Mevacor), Pitavastatin (Livalo), Pravastatin (Pravachol), Rosuvastatin (Crestor), Simvastatin (Zocor), Nicotinic acids: Niacin, Cholesterol absorption inhibitor: Ezetimibe (Zetia), Combination statin and cholesterol absorption inhibitors: Ezetimibe/Simvastatin (Vytorin), which lower bad cholesterol. Digitalis preparations include: Digoxin (Lanoxin), which increases the force of the heart's beat and slows a fast heart rate. Diuretics include: Acetazolamide (Diamox), Amiloride (Midamor), Bumetanide (Bumex), Chlorothiazide (Diuril), Chlorthalidone (Hygroton), Furosemide (Lasix), Hydro-chlorothiazide (Esidrix, Hydrodiuril), Indapamide (Lozol), Metalozone (Zaroxolyn), Spironolactone (Aldactone), Torsemide (Demadex), which cause the body to rid itself of excess fluids and sodium through urination and helps reduce the heart's workload. Vasodilators include: Isosorbide dinitrate (Isordil), Isosorbide mononitrate (Imdur), Hydralazine (Apresoline), Nitroglycerin (Nitro Bid, Nitro Stat), Minoxidil. dilates) the blood vessels so the blood flows more easily and the heart doesn't have to work as hard and decreases blood pressure. A category of vasodilators called nitrates increases the supply of blood and oxygen to the heart while reducing its workload which can ease chest pain (angina), e.g., nitroglycerin is available as a pill to be swallowed or absorbed under the tongue (sublingual), a spray, and as a topical application (cream).

For neurological conditions include one of more of the following can be administered to the subject based on the personalized risk assessment: Drugs often prescribed by neurologists include: anticonvulsants, or antiepileptic drugs which prevent/treat abnormal electrical activity in the brain. Drugs to treat patients with dementia or Parkinson's disease, and antidepressants, beta blockers, and blood thinners. Some of the more commonly medications neurologists prescribe include: Levetiracetam which is used to treat epilepsy. Gabapentin which is used to treat partial seizures, nerve pain from shingles and restless leg syndrome. Topiramate which is used to treat various mood and eating disorders and helps in substance abuse therapy. Lamotrigine which is used to treat the partial seizures, primary generalized tonic-clonic seizures, bipolar I disorder maintenance. Carbidopa-Levodopa which is used in the management and treatment of Parkinson disease (PD). Carbidopa is indicated for combination use with levodopa (L-dopa) for the treatment of motor symptoms occurring in Parkinson disease (PD) and post-encephalitic parkinsonism. Donepezil HCl which is used to treat dementia associated with mild, moderate, or severe Alzheimer's disease, and it can improve cognition and behavior, thereby alleviating certain symptoms. Sumatriptan Succinate which is used to treat migraine headaches. Used when headache symptoms first start, not used to prevent headaches. Oxcarbazepine which is used to treat epilepsy (partial seizures). Amitriptyline HCl which is used to increase the level of specific chemicals in your brain, which improves your depression. Memantine HCl which is used to in Alzheimer's disease treatment as a non-competitive modern-affinity strong voltage-dependent N-methyl-D-aspartate receptor antagonist. The therapeutic abilities of memantine can also be used to of various psychiatric illnesses such as autism spectrum disorder, binge eating disorder, and attention-deficit/hyperactivity disorder.

A person of skill in the art would readily recognize that steps of various above-described methods can be performed by one or more programmed computers, each having one or more computer processors. Herein, some embodiments are also intended to cover program storage devices, e.g., digital data storage media, which are machine or computer-readable and encode machine-executable or computer-executable programs of instructions, wherein the instructions perform some or all of the steps of said above-described methods. The program storage devices may be, e.g., digital memories, magnetic storage media such as magnetic disks and magnetic tapes, hard drives, or optically readable digital data storage media. The embodiments are also intended to cover computers programmed to perform said steps of the above-described methods.

A risk score of the present invention may be calculated with an algorithm using well-known statistical analysis techniques. Non-limiting examples of statistical analysis techniques that may be used to calculate the risk score include cross-correlation, Principal Components Analysis (PCA), factor rotation, Logistic Regression (Log Reg), Linear Discriminant Analysis (LDA), Eigengene Linear Discriminant Analysis (ELDA), Support Vector Machines (SVM), Random Forest (RF), Recursive Partitioning Tree (RPART), related decision tree classification techniques, Shrunken Centroids (SC), StepAIC, Kth-Nearest Neighbor, Boosting, Decision Trees, Neural Networks, Bayesian Networks, Support Vector Machines, and Hidden Markov Models, Linear Regression or classification algorithms, Nonlinear Regression or classification algorithms, analysis of variants (ANOVA), hierarchical analysis or clustering algorithms; hierarchical algorithms using decision trees; kernel based machine algorithms such as kernel partial least squares algorithms, kernel matching pursuit algorithms, kernel Fisher's discriminate analysis algorithms, or kernel principal components analysis algorithms. In preferred embodiments, the risk score may be calculated using a random forest algorithm using the concentrations of three or more sample analytes in the panel of biomarkers. In an exemplary embodiment, the risk score is calculated as described in the examples.

The functions of the various elements shown in the figures, including any functional blocks labeled as “modules”, may be provided through the use of dedicated hardware as well as hardware capable of executing software in association with the appropriate software. When provided by a processor, the functions may be provided by a single dedicated processor, by a single shared processor, or by a plurality of individual processors, some of which may be shared. Moreover, explicit use of the term “module” should not be construed to refer exclusively to hardware capable of executing software, and may implicitly include, without limitation, digital signal processor (DSP) hardware, network processor, application-specific integrated circuit (ASIC), field-programmable gate array (FPGA), read-only memory (ROM) for storing software, random access memory (RAM), and nonvolatile storage. Other hardware, conventional and/or custom, may also be included.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least 1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

What is claimed is:

1. A method of determining a personalized risk assessment for a subject comprising:
receiving and normalizing one or more biometric data streams of the subject;
using a processor and a multimodal machine learning algorithm to establish personalized benchmark profile and determine personalized risk assessment:
identifying for each of the normalized biometric data stream values for: minimum, maximum, delta, mean, median, and average minimum and average maximum boundaries;
reducing noise from non-essential features by cross feature linear regression analysis to obtain a dimensionality reduction; and
stratifying each of two or more normalized biometric data stream values into nodes with a high or maximum predictive value and minimum predictive value, wherein nodes with high or maximum predictive value are selected to calculate the personalized risk assessment;
using the nodes with high or maximum predictive value to identify biometric data values for the personalized risk assessment; and
administering conducting at least one of: post-disease recovery, post-procedure monitoring, diagnostic tests, or treatment to the subject based on the personalized risk assessment.

2. The method of claim 1, wherein the diagnostic tests are selected based on the personalized risk assessment selected from obtaining biological samples to test for bacterial, viral, fungal, or parasitic infection, metabolic panels, phenotyping panels, genotyping panels, microbiome panels, autoimmune disease or condition testing.

3. The method of claim 1, wherein the treatment is selected from one or more cardiovascular drugs, antimicrobial drugs, wherein the disease, disorder or condition is an infectious disease or disorder, and an autoimmune disease.

4. The method of claim 1, wherein normalizing the one or more biometric data streams by categorizing the biometric data stream into three categories: maximum frequency biometric data, medium frequency biometric data, and minimum frequency biometric data and selecting data values for a fixed or variable time interval.

5. The method of claim 1, wherein the maximum frequency biometric data, medium frequency biometric data, and minimum frequency biometric data is selected from at least one of: daytime and nighttime, activity (1G, 2G, 3G acceleration), heart rate, heart rate variability, resting heart rate, blood oxygen saturation, or motion sensors that measure along at least one of an X, Y, or Z axis.

6. The method of claim 1, wherein the dimensionality reduction is selected from linear regression, z-score, p-value, gaussian, normal distribution, and statistical method.

7. The method of claim 6, further comprising stratifying each of two or more normalized biometric data stream values into nodes with high or maximum predictive value and minimum predictive value.

8. The method of claim 1, further comprising, after using the nodes with high or maximum predictive value to identify biometric data values for the personalized risk assessment, then determining:
if the one or more nodes meet or exceed a predetermined risk assessment value threshold, then using the nodes for the personalized risk assessment, or
if the one or more nodes is below the predetermined risk assessment value threshold, then repeating the step of selecting one or more nodes using a different statistical model until the statistical significance meets or exceeds the personalized risk assessment threshold.

9. The method of claim 1, further comprising, after using the nodes with high or maximum predictive value to identify biometric data values for the personalized risk assessment, then determining:
if the one or more nodes meet or exceed a predetermined risk assessment value threshold, then using the nodes for the personalized risk assessment, or
if the one or more nodes is below the predetermined risk assessment value threshold, then:
repeating the step of receiving and normalizing one or more biometric data streams of the subject;
repeating the step of identifying for each of the normalized biometric data stream values for: minimum, maximum, delta, mean, median, and average minimum and average maximum boundaries;
repeating the step of reducing noise in each of the normalized biometric data stream values by:
repeating the step of stratifying each of two or more normalized biometric data stream values into nodes with high or maximum or maximum predictive value and minimum predictive value, wherein nodes with high or maximum or maximum predictive value are selected to calculate the personalized risk assessment; or
repeating the step of using the nodes with high or maximum predictive value to identify biometric data values for the personalized risk assessment, until:
the statistical significance meets or exceeds the personalized risk assessment threshold.

10. The method of claim 1, wherein the one or more biometric data streams are obtained from a wearable device, one or more sensors transiently or permanently attached to or inserted into the subject, or sensors in a room, chair or bed.

11. The method of claim 1, further comprising placing the subject into a cohort of patients with one or more medical conditions or diseases of the subject.

12. The method of claim 1, wherein the one or more data streams are obtained from at least one of: one or more wearable devices, one or more medical beds, one or more O₂ sensors, one or more blood pressure sensors, one or more electrocardiogram (ECG), accelerometers, or gyroscopes.

13. The method of claim 1, wherein the one or more biometric sensor devices is selected from O₂ sensor(s), accelerometer(s), gyroscope(s), electrocardiogram, accelerometer(s), gyroscope(s), heart rate monitor(s), or pulse monitor(s).

14. The method of claim 1, wherein the biometric data further identifies blood pressure, step count, active energy burned, basal energy burned, sleep status, temperature, respiratory rate, EKG, posture, or fall detection.

15. The method of claim 1, wherein the biometric data is categorized into three categories: high frequency, medium frequency, and low frequency;
category I: high frequency with distinct patterns during day vs. night;
category II: medium frequency data points with some daily data points;
category III: low frequency with no or a few data points per day;
and the personalized benchmark-based outliers, statistic-based features selected from at least one of:
heart rate outliers:
day upper outliers: the heart rate data points higher than an established day upper threshold of the subject;
day lower outliers: the heart rate data points lower than an established day upper threshold of the subject;
night upper outliers: the heart rate data points higher than an established day upper threshold of the subject;
night lower outliers: the heart rate data points lower than an established day upper threshold of the subject;
physical activities outlier-based features:
at least one of: day 1 g, 2 g, or 3 g upper outliers;
at least one of: day 1 g, 2 g, or 3 g lower outliers;
at least one of: night 1 g, 2 g, or 3 g upper outliers;
at least one of: night 1 g, 2 g, or 3 g lower outliers; and
statistics: min, mean, median, max, sum.

16. The method of claim 15, wherein the one or more temporal segments in the Category I data streams are selected from 0.1, 1, 5, 10, 15, 20, 25, 30, 40, 50, 60, 70, 75, 80, 90, 100 milliseconds, 0.1, 1, 5, 10, 15, 20, 25, 30, 40, 50, 60, 70, 75, 80, 90, 100 seconds, 0.1, 1, 5, 10, 15, 20, 25, 30, 40, 50, 60, 70, 75, 80, 90, to 100 minutes; the one or more temporal segments in the Category II data streams are selected from 0.1, 1, 5, 10, 15, 20, 25, 30, 40, 50, 60, 70, 75, 80, 90, 100 hours, 0.1, 1, 5, 10, 15, 20, 25, 30, 40, 50, 60, 70, 75, 80, 90, 100 days, 0.1, 1, 5, 10, 15, 20, 25, 30, 40, 50, 52, 60, 70, 75, 80, 90, to 100 weeks; and the one or more temporal segments in the Category III data streams are selected from 0.1, 1, 5, 10, 15, 20, 25, 30, 40, 50, 60, 70, 75, 80, 90, 100 days, 0.1, 1, 5, 10, 15, 20, 25, 30, 40, 50, 52, 60, 70, 75, 80, 90, 100 weeks, 0.1, 1, 5, 10, 15, 20, 25, 30, 40, 50, 60, 70, 75, 80, 90, to 100 months.

17. The method of claim 15, wherein the one or more temporal segments in the Category I, II, or III data streams can be fixed, variable, periodic, or aperiodic.

18. The method of claim 15, wherein the biometric data is selected from at least one of: nightmore3G_median; daymore1G_min; daymore2G_min; daymore3G_min; nightmore1G_min; nightmore1G_delta_min; nightmore1G_lower_outlier; nightmore1G_lower_outlier_percentage; nightmore2G_min; nightmore2G_delta_min; nightmore2G_lower_outlier; nightmore2G_lower_outlier_percentage; nightmore3G_min; nightmore3G_delta_min; nightmore3G_lower_outlier; nightmore3G_lower_outlier_percentage; daymore1G_delta_min; daymore1G_lower_outlier; daymore1G_lower_outlier_percentage; daymore2G_delta_min; daymore2G_lower_outlier; daymore2G_lower_outlier_percentage; daymore3G_delta_min; daymore3G_lower_outlier; daymore3G_lower_outlier_percentage; bloodOxygenSaturation_delta_max; bloodOxygenSaturation_upper_outlier; bloodOxygenSaturation_upper_outlier_percentage; nightmore2G_median; daymore3G_median; bloodOxygenSaturation_max; bloodOxygenSaturation_lower_outlier; bloodOxygenSaturation_outlier; dayheartRate_delta_min; bloodOxygenSaturation_delta_min; heartRateVariability_upper_outlier; dayheartRate_min; nightmore1G_median; heartRateVariability_lower_outlier_percentage; bloodOxygenSaturation_min; heartRateVariability_outlier; dayheartRate_max; nightmore1G_upper_outlier_percentage; nightmore1G_outlier_percentage; heartRateVariability_delta_max; nightmore1G_upper_outlier; nightmore1G_outlier; dayheartRate_delta_max; nightheartRate_min; restingHeartRate_upper_outlier_percentage; heartRateVariability_max; heartRateVariability_delta_min; nightheartRate_delta_min; restingHeartRate_delta_max; nightheartRate_mean; nightmore2G_delta_max; nightheartRate_upper_outlier_percentage; nightmore2G_max; dayheartRate_upper_outlier; nightheartRate_median; nightmore1G_delta_max; nightmore1G_max; restingHeartRate_max; heartRateVariability_lower_outlier; daymore3G_delta_max; restingHeartRate_median; daymore3G_max; dayheartRate_lower_outlier; restingHeartRate_upper_outlier; nightmore3G_mean; daymore2G_delta_max; nightheartRate_delta_max; nightheartRate_lower_outlier; daymore2G_max; restingHeartRate_mean; nightmore2G_mean; dayheartRate_outlier; nightmore3G_delta_max; dayheartRate_upper_outlier_percentage; nightmore3G_max; nightheartRate_max; daymore1G_delta_max; nightmore2G_upper_outlier_percentage; nightmore2G_outlier_percentage; nightmore1G_sum; nightmore3G_sum; nightmore2G_upper_outlier; nightmore2G_outlier; nightmore1G_mean; nightmore2G_sum; heartRateVariability_upper_outlier_percentage; nightheartRate_lower_outlier_percentage; nightheartRate_outlier; daymore2G_median; nightheartRate_upper_outlier; nightmore3G_upper_outlier_percentage; nightmore3G_outlier_percentage; daymore1G_upper_outlier; daymore1G_outlier; nightmore3G_upper_outlier; nightmore3G_outlier; restingHeartRate_lower_outlier; heartRateVariability_min; restingHeartRate_min; restingHeartRate_lower_outlier_percentage; bloodOxygenSaturation_median; heartRateVariability_outlier_percentage; daymore1G_max; daymore1G_sum; daymore1G_median; restingHeartRate_outlier; dayheartRate_median; daymore3G_upper_outlier; daymore3G_outlier; nightheartRate_outlier_percentage; daymore3G_sum; bloodOxygenSaturation_lower_outlier_percentage; bloodOxygenSaturation_outlier_percentage; dayheartRate_mean; restingHeartRate_delta_min; heartRateVariability_mean; heartRateVariability_median; daymore1G_upper_outlier_percentage; daymore1G_outlier_percentage; daymore3G_upper_outlier_percentage; daymore3G_outlier_percentage; daymore2G_sum; daymore3G_mean; daymore2G_upper_outlier; daymore2G_outlier; daymore1G_mean; dayheartRate_lower_outlier_percentage; daymore2G_mean; daymore2G_upper_outlier_percentage; daymore2G_outlier_percentage; bloodOxygenSaturation_mean; dayheartRate_outlier_percentage; or restingHeartRate_outlier_percentage.

19. The method of claim 1, wherein a circadian boundary detection used hourly heart rate and acceleration via k means.

20. The method of claim 1, further comprising determining an outlier counting value per circadian segment, wherein biometric outliers are counted separately for each morning segment, daytime segment and night segment relative to one or more personalized thresholds.

21. The method of claim 1, further comprising using a dynamic benchmark window to generates a dynamic physiological benchmark by combining an initialization window comprising an early segment of user data with a rolling, continuously updated benchmark window, to provide one or more flexible and progressively personalized comparison metrics for risk detection.

22. The method of claim 1, further comprising using one or more nodes comprising one or more related features to evaluate and stratify the data using one or more statistical tests selected from t tests or z scores to determine a predictive strength.

23. The method of claim 1, further comprising calculating one or more personalized feature contribution scores that computes a relative weight of one or more biometric data or datastreams during short-term or long-term conditions to rank features by overall impact on predictions across a part of or all of a dataset.

24. A non-transitory computer-readable medium for determining a personalized risk assessment for a subject comprising instructions stored thereon, that when executed on a processor, perform the steps of:
receiving an electronic communication containing one or more biometric data streams of the subject;
using a processor and a multimodal machine learning algorithm to establish personalized benchmark profile and determine personalized risk assessment by:
identifying for each of the normalized biometric data stream values for: minimum, maximum, delta, mean, median, and average minimum and average maximum boundaries;
reducing noise from non-essential features by cross feature linear regression analysis to obtain a dimensionality reduction; and
stratifying each of two or more normalized biometric data stream values into nodes with a high or maximum predictive value and minimum predictive value, wherein nodes with high or maximum predictive value are selected to calculate the personalized risk assessment;
using the nodes with high or maximum predictive value to identify biometric data values for the personalized risk assessment; and
administering conducting at least one of: post-disease recovery, post-procedure monitoring, diagnostic tests, or treatment to the subject based on the personalized risk assessment.

25. A computer-implemented method for determining a personalized risk assessment for a subject, the method comprising:
receiving an electronic communication containing one or more biometric data streams of the subject;
normalizing the dataset;
using a processor and a multimodal machine learning algorithm to establish personalized benchmark profile and determine personalized risk assessment:
identifying for each of the normalized biometric data stream values for: minimum, maximum, delta, mean, median, and average minimum and average maximum boundaries;
reducing noise from non-essential features by cross feature linear regression analysis to obtain a dimensionality reduction; and
stratifying each of two or more normalized biometric data stream values into nodes with a high or maximum predictive value and minimum predictive value, wherein nodes with high or maximum predictive value are selected to calculate the personalized risk assessment;
using the nodes with high or maximum predictive value to identify biometric data values for the personalized risk assessment; and
administering conducting at least one of: post-disease recovery, post-procedure monitoring, diagnostic tests, or treatment to the subject based on the personalized risk assessment.
